(19) United States
(12) Patent Application Publication
(10) Pub. No.: US 2024/0308943 A1
(43) Pub. Date: Sep. 19, 2024

(54) EXTRACTION, SEPARATION AND PURIFICATION OF CANNABINOIDS FROM CANNABIS STAIVA AND OTHER MARIJUANA BIOMASS

(71) Applicant: Trevor Percival Castor, Arlington, MA (US)

(72) Inventor: Trevor Percival Castor, Arlington, MA (US)

(73) Assignee: Aphios Corporation, Arlington, MA (US)

(21) Appl. No.: 18/039,480

(22) PCT Filed: Nov. 29, 2021
(86) PCT No.: PCT/US21/61038

§ 371 Date: May 30, 2023

Related U.S. Application Data
(60) Provisional application No. 63/118,960, filed on Nov. 29, 2020.

(51) Int. Cl. C07C 37/00; B01D 11/02; B01D 15/40; B01D 15/42; C07C 51/48; C07D 311/80; G01N 30/74

(52) U.S. Cl. CPC C07C 37/004; B01D 11/0203; B01D 11/0207; B01D 11/0219; B01D 11/028; B01D 11/0288; B01D 11/0292; B01D 15/40; B01D 15/426; C07C 51/48; C07D 311/80; G01N 30/74; C07C 2601/16

(57) ABSTRACT

This invention is for improved extracting, separating, and manufacturing pharmaceutical grade CBD and other cannabinoids following current Good Manufacturing Practices (cGMP) of the US FDA for use in clinical trials for central nervous system (CNS) and peripheral nervous system (PNS) disorders such as pain, opioid use disorder, anxiety, epilepsy, nausea and vomiting, Multiple sclerosis and other indications by the National Institute of Health (NIH) and other researchers. This invention first established optimum conditions for the selective SuperFluids™ [supercritical fluids and near-critical fluids with or without polar co-solvents such as alcohols] fractionation of Cannabis sativa to isolate CBD, CBDA, Δ9-THC, Δ9-THCA and other cannabinoids Δ9-THC; then defined SuperFluids™ chromatographic purification conditions for the further purification of CBD, CBDA, Δ9-THC, Δ9-THCA with absolute purities >98.5%. This invention is for methods and apparatus to manufacture pharmaceutical-grade CBD and CBDA (98.5% to 100% with <0.3% Δ9-THC) from Cannabis sativa, hemp and marijuana utilizing SuperFluids™ selective extraction and chromatographic purification technologies, and the manufacture of other bioactive cannabinoids such as Δ9-THC, Δ9-THCA, Δ8-THCA, CBG, CBGA, CBC, CBN and their mixtures for future preclinical and clinical research, and their therapeutic use.

### GOVERNMENT SUPPORT

Research leading to this invention was in part funded by the National Institute on Drug Abuse and the National Cancer Institute, National Institutes of Health, Bethesda, MD, USA.

### FIELD OF THE INVENTION

This invention relates to methods for making and separating cannabinoids from Cannabis sativa, hemp and other Marijuana biomass. The methods feature supercritical, critical and near-critical fluids with and without polar cosolvents.

### REFERENCES TO OTHER PATENTS

This application discloses a number of improvements and enhancements to methods and apparatus for isolating therapeutic compositions from source materials disclosed in U.S. Pat. No. 5,750,709 to Castor, and methods and apparatus for extracting Taxol from source materials in U.S. Pat. No. 5,440,055 to Castor, which are hereby incorporated by reference in their entirety.

### BACKGROUND OF THE INVENTION

The legitimate use of marijuana for several medical indications has far outpaced the medical and clinical evaluation of marijuana and specific cannabinoids for different medical uses. In 1997, the National Institutes of Health convened an Ad Hoc Expert Panel to discuss current knowledge of the medical uses of Cannabis. The report discussed the importance of other cannabinoids and their potential interaction effects upon THC, stating: “Varying proportions of other cannabinoids, mainly cannabidiol (CBD) and cannabinol (CBN), are also present in Cannabis, sometimes in quantities that might modify the pharmacology of THC or cause effects of their own. CBD is not psychoactive but has significant anticonvulsant, sedative, and other pharmacological activity likely to interact with THC.” The Institute of Medicine (IOM, 1999) concluded that scientific data indicate the potential therapeutic value of cannabinoid drugs, primarily 66 9-THC, for pain relief, control of nausea and vomiting, and appetite stimulation and clinical trials of cannabinoid drugs for symptom management should be conducted.

Medical marijuana is now approved in 36 states and the District of Columbia for several medical conditions such as cachexia, cancer, chronic pain, epilepsy and other disorders characterized by seizures, glaucoma, HIV, AIDS, Multiple Sclerosis, muscle spasticity and nausea. Progress has been made on several fronts on the use of cannabinoids for medical use such as Charlotte's Web (CW) being used for childhood epilepsy through ad hoc development by patient advocacy groups. Sativex® (GW Pharmaceuticals, England), a drug containing equal proportions of Δ9-THC and CBD, is approved as a second-line treatment for Multiple Sclerosis (MS) associated spasticity in Canada, New Zealand and several European countries.

The FDA has recently approved Epidiolex® (GW Pharmaceuticals, England), which contains a purified form of the drug substance cannabidiol (CBD) for the treatment of seizures associated with Lennox-Gastaut syndrome or Dravet syndrome in patients 2 years of age and older. The ready availability of pharmaceutical-grade CBD and standardized cannabinoid (CB) products, manufactured following cGMP guidelines, will facilitate clinical evaluation by NIH investigators and other researchers for epilepsy, MS and other CNS diseases. The developed process will also be utilized for the manufacturing of Δ9-THC, already in use for cancer pain and nausea and AIDS-related cachexia, and other cannabinoids in development such as CBDA, Δ9-THCA, Δ8-THCA, Δ8-THC, CBGA, CBG, CBC and CBN.

This invention is for the extraction, separation, purification and manufacturing of pharmaceutical-grade CBD and other cannabinoids for clinical evaluation by the NIH and other pharmaceutical companies for Multiple Sclerosis and other CNS and PNS diseases, and standardized cannabinoid (CB) products for use by medical marijuana dispensaries in Massachusetts and other states for childhood epilepsy and other diseases.

### SUMMARY OF THE INVENTION

Embodiments of the present invention are directed to methods of making and separating and purifying cannabinoids such as CBD, CBDA, Δ9-THC (THC), Δ9-THCA (THCA), Δ9-THCA, Δ8-THCA, Δ8-THC, CBGA, CBG, CBC and CBN, and others from Cannabis sativa and other Marijuana biomass. These methods employ the use of supercritical, near-critical or critical fluids with or without small molar quantities of polar entrainers or cosolvents such. Hereinafter, these fluids are referred to as SuperFluids™ or SFS™.

As a further aspect of the invention, a mixture of cannabinoids is first extracted from Cannabis sativa biomass utilizing SuperFluids™ and deposited onto the head of a chromatographic column containing a solid phase. The eluant through the solid phase is collected in fractions and consist of non-ionic or neutral cannabinoids. The extraction column containing Cannabis sativa biomass is then bypassed and the composition of the SuperFluids™ is changed to separate and elute purified cannabinoids from the chromatographic column.

Preferably, the SuperFluids™ is carbon dioxide that is held at a temperature of 20-60° C., at a pressure of 1,000 to 5,000 psig. Preferably, the carbon dioxide has a modifier, in the sense that the modifier is carried in the carbon dioxide in the nature of a dissolved constituent. A preferred modifier is an alcohol, such as methanol or ethanol.

Preferably, the composition of the first SuperFluids used to extract the mixture of cannabinoids is 100% carbon dioxide at a temperature of 20-60° C., at a pressure of 1,000 to 5,000 psig.

Preferably, the composition of the first SuperFluids used to extract the mixture of cannabinoids is 100% carbon dioxide at a temperature of 50° C., at a pressure of 3,000 psig.

In another embodiment of the invention, the biomass is then extracted after the first step with SuperFluids that consist of 95% carbon dioxide and 5% cosolvent such as ethanol or methanol.

In one embodiment of this invention, the cannabinoids extracted by the first SuperFluids are deposited onto the head of a chromatographic column that selectively adsorbs certain cannabinoids such as carboxylated and charged cannabinoids.

Preferably, the non-carboxylated and neutral cannabinoids that flow through the column are collected in fractions resulting in purification of certain cannabinoids.

In another embodiment of this invention, the column containing the Cannabis or hemp biomass is bypassed and a second SuperFluids composition is used to chromatograph the adsorbed cannabinoids through and off the chromatographic column separating and purifying the adsorbed cannabinoids.

Preferably, the composition of the second SuperFluids used to separate and elute the cannabinoids from the chromatographic column is a mixture of carbon dioxide and a cosolvent at a temperature over the range of 20-60° C. and a pressure over the range of 1,000 to 5,000 psig.

Preferably, the composition of the second SuperFluids used to separate and elute the cannabinoids from the chromatographic column is a mixture of carbon dioxide and a cosolvent at room temperature of 25° C. and a pressure of 3,000 psig.

Preferably, the cosolvent used with the second SuperFluids is an alcohol such as methanol or ethanol.

Preferably, the cosolvent used with the second SuperFluids is ethanol.

Preferably, the composition of the second SuperFluids is a gradient of cosolvent and CO₂ starting at 0.2% cosolvent:99.8% CO₂ to 2% cosolvent:98% CO_{2.}

Preferably, the composition of the second SuperFluids is an isocratic mixture of cosolvent and CO₂ of 1.5% cosolvent:98.5% CO₂.

Preferably, the Cannabis sativa and Marijuana biomass is ground into a fine powder to increase surface are to volume area and increase extraction efficiency.

Preferably, the Cannabis sativa, hemp and Marijuana biomass is dried at a low temperature to preserve the integrity of the cannabinoids and to remove the mass transfer resistance to extraction caused by water.

Preferably, the Cannabis sativa, hemp and Marijuana biomass is dried with a stream of warm air or N₂ at temperatures in the range of 25° C. to 60° C.

Preferably, the Cannabis sativa, hemp and Marijuana biomass is dried with a stream of warm air or N₂ at temperatures at 40° C.

Preferably, the Cannabis sativa, hemp and Marijuana biomass is dried with a stream of N₂ at a temperature at 40° C.

Preferably, the solid phase in the chromatographic column has a particle size of 10 to 100 microns and consists of C18, C10, C8 or silica.

Preferably, the solid phase is silica.

Preferably, the solid chromatographic phase is dehydrated of water and activated for adsorption of analytes.

In another embodiment of the invention, thermodynamic analyses are performed to match the solubility parameters of targeted cannabinoids with the solubilization selectivity of SuperFluids to guide experimental analysis and manufacturing.

In another embodiment of the invention, the back-pressure regulator is continually flushed with a high pressure, low volume stream of cosolvent such as ethanol or methanol during extraction and chromatographic purification operations in order to clear the back-pressure regulator of any precipitated solutes and provide a low heat source to prevent freezing of the back-pressure regulator due to Joule-Thompson cooling effects.

These and other features and advantages will be apparent to those skilled in the art upon reading the examples, detailed description and viewing the drawings briefly described below.

### BRIEF DESCRIPTION OF THE DRAWINGS

FIG. 1 depicts in schematic form an apparatus embodying features of the present invention;

FIG. 2 depicts a Process Flow Diagram of a Research-Scale SuperFluids™ Extraction and Fractionation (SFS-CXF) Apparatus;

FIG. 3 depicts a Process Flow Diagram of a Bench-Top SuperFluids™ Extraction and Purification (SFS-CXP) Apparatus;

FIG. 4 depicts a Process Flow Diagram of a SuperFluids™ Extraction and Purification (SFS-CXP) Semi-Works Plant;

FIG. 5 depicts a Standard Regression Curve for CBD (cannabidiol);

FIG. 6 depicts a Standard Regression Curve for Δ9-THC (Δ9-tetrahydrocannabinol);

FIG. 7 depicts a Standard Regression Curve for Δ9-THCA (Δ9-tetrahydrocannabinoic acid);

FIG. 8 depicts a Standard Regression Curve for CBN (cannabinol);

FIG. 9 shows the chemical structure of CBD;

FIG. 10 shows chemical structure of Δ9-THC;

FIG. 11 depicts Hildebrand Solubility Parameter, δ, of SuperFluids™ as a Function of Temperature and Pressure;

FIG. 12 shows a HPLC Chromatogram of SuperFluids™ Extraction of Heat-Treated Cannabis [MAJ-21-3S];

FIG. 13 depicts SuperFluids™ CXP of Heat-Treated Cannabis [MAJB-4];

FIG. 14 shows a HPLC Chromatogram of SuperFluids™CXP of Heat-Treated Cannabis[MAJB-4-F2];

FIG. 15 shows a HPLC Chromatogram of SuperFluids™CXP of Untreated Cannabis[MAJB-3-F8];

FIG. 16 shows a HPLC Chromatogram of CBDA-CBD-I-24-11 at 1×10 Dilution;

FIG. 17 shows an UV Spectra of CBDA-CBD-I-24-11 at 1×10 Dilution;

FIG. 18 depicts Cumulative Elution Profiles of CBDA Fractionated from Uunheated Cannabis sativa Biomass with SuperFluids as a Function of Time;

FIG. 19 shows the results of CBD-II-57 for the SFS-CXP of high CBD content Cannabis sativa [unground and unheated]: Extraction with SFS CO₂ at 3,000 psig and 50° C., Deposition on Silica [heat activated] followed by Supercitical Fluid Chromatography using SFS CO₂:methanol at 3,000 psig and 25° C.;

FIG. 20 shows the results of CBD-II-59 for the SFS-CXP of high CBD content Cannabis sativa [unground and unheated]: Extraction with SFS CO₂ at 3,000 psig and 50° C., Deposition on Silica [not heat activated] followed by Supercitical Fluid Chromatography using SFS CO₂:methanol at 3,000 psig and 25° C.;

FIG. 21 shows the results of CBD-II-67 for the SFS-CXP of high CBD content Cannabis sativa [ground and oven-dried at 40° C.]: Extraction with SFS CO₂ at 3,000 psig and 50° C., Deposition on two Silica columns in series [heat treated at 130° C.] followed by Supercitical Fluid Chromatography using SFS CO₂:methanol at 3,000 psig and 25° C.;

FIG. 22 shows an HPLC Chromatographic Scan of CBD-II-67-09 (CBD);

FIG. 23 shows an HPLC Chromatographic Scan of CBD-II-67-20 (49-THC);

FIG. 24 shows an HPLC Chromatographic Scan of CBD-II-67-24 (CBDA);

FIG. 25 shows the results of CBD-II-80 for In Situ Ground Cannabis Biomass Drying with N₂ gas [10.5 LPM at 100 psig and 60° C.];

FIG. 26 shows the results of CBD-II-81 Comparing the Absolute Purity (%) and Cannabinoid Content (mg) by Fraction Number; and

FIG. 27 shows the results of CBD-II-92 Comparing Relative Purity (%) and Cannabinoid Content (mg) by Fraction Number.

### DETAILED DESCRIPTION OF PREFERRED EMBODIMENTS

In the United States, medical marijuana is now approved in 36 states and the District of Columbia, Guam, Puerto Rico and U.S. Virgin Islands for several medical conditions such as cachexia, cancer, chronic pain, epilepsy and other disorders characterized by seizures, glaucoma, HIV, AIDS, Multiple Sclerosis, muscle spasticity and nausea. Medical marijuana use is on the ballot in additional states, and it is also approved for recreational use in 18 states including Massachusetts, Washington state and California. The legitimate use of marijuana for several medical indications has far outpaced the medical and clinical evaluation of marijuana and specific cannabinoids for different medical uses. In 1997, the National Institutes of Health convened an Ad Hoc Expert Panel to discuss current knowledge of the medical uses of Cannabis. The report discussed the importance of other cannabinoids and their potential interaction effects, stating: “Varying proportions of other cannabinoids, mainly cannabidiol (CBD) and cannabinol (CBN), are also present in Cannabis, sometimes in quantities that might modify the pharmacology of THC or cause effects of their own. CBD is not psychoactive but has significant anticonvulsant, sedative, and other pharmacological activity likely to interact with THC.” The Institute of Medicine (IOM, 1999) concluded that scientific data indicate the potential therapeutic value of cannabinoid drugs, primarily Δ9-THC, for pain relief, control of nausea and vomiting, and appetite stimulation and clinical trials of cannabinoid drugs for symptom management should be conducted. In 2003, NIH was awarded a United States patent for use of cannabinoids as antioxidants and neuroprotectants (Hampson et al., 2003).

Progress has been made on several fronts on the use of cannabinoids for medical use, both through rigorous clinical evaluation of Δ9-THC for cancer pain and nausea and cachexia associated with HIV/AIDS, Δ9-THC/CBD mixtures for Multiple Sclerosis and muscle spasticity, and ad hoc development by localized medical marijuana dispensaries and patient advocacy. The latter is especially true of Charlotte's Web (CW) being used for childhood epilepsy. The strain was named for 5-year-old Charlotte Figi, who had been suffering from a rare disorder called Dravet's syndrome, which caused her to have as many as 300 grand mal seizures a week. Charlotte used a wheelchair, went into repeated cardiac arrest, could barely speak and had flat-lined at least 3 times by 2012. Two years later, Charlotte is largely seizure-free and able to walk, talk and feed herself after taking oil infused with a high CBD Cannabis strain with low Δ9-THC content (CBS News, 2014).

Even with lack of well-controlled clinical evidence, families have been migrating to Colorado to seek treatment for their children. Seeds of the high-CBD hemp have migrated to other states. Recently, Utah's Governor signed “Charlee's Law,” a hemp supplement bill allowing epilepsy patients access to cannabis oils, after six-year old Charlee Jordan who suffered from Late Infant Batten Disease, a terminal inherited disorder of the nervous system that leads to seizures, and loss of vision and motor skills (The Salt Lake Tribune, 2014). More than 3 million people in North America, 6 million in Europe and 50 million worldwide have epilepsy with highest prevalence for children below five years of age and the elderly with about 30% of patients non-responsive to traditional anti-epileptic drugs (WHO, 2007). The Global Epilepsy Drug Market size was estimated at USD $3.7 billion in 2020 and expected to reach USD $4.0 billion in 2021. at a CAGR 6.89% to reach USD $5.6 billion by 2026 (Reportlinker.com, Oct. 18, 2021).

Sativex® (GW Pharmaceuticals, England), a drug containing equal proportions of 49-THC and CBD, is approved as a second-line treatment for Multiple Sclerosis (MS) associated spasticity in Canada, New Zealand and several European countries. In October 2013, the Food and Drug Administration approved clinical testing of GW Pharmaceuticals' marijuana-derived drug that is CBD-based. MS is a demyelinating and neurodegenerative disease of the CNS, which is one of the main causes of irreversible neurologic disability in young adults. MS is notoriously heterogeneous in terms of its clinical manifestations and evolution, as well as in terms of its immunopathological substrates. The disease affects 2.5 million people worldwide, of which 1,000,000 are in the USA and 500,000 in the EU. According to the Cleveland Clinic, MS-related health care costs are thought to be over $10 billion per year in the United States. Despite being the most common human primary demyelinating disease of the CNS, there is no satisfactory treatment as yet for MS, and there is a clear need for the development of agents able to treat this progressive disorder.

The development of a manufacturing process for cannabinoid pharmaceuticals such as CB and CBD is significant because they could be used for studying the physiological effects and the therapeutic value of cannabinoids in humans, potentially leading to new therapeutic agents that could benefit a number of patients. The ready availability of pharmaceutical-grade CBD and standardized CB products, following cGMP guidelines, will facilitate clinical evaluation by NIH investigators and other researchers for epilepsy, MS and other CNS and PNS diseases. The developed process can also be utilized for the manufacturing of Δ9-THC, already in use for cancer pain and nausea and AIDS-related cachexia, and other cannabinoids in development.

Aspects of the present invention employ materials known as supercritical, critical or near-critical fluids with or without small molar concentrations of polar cosolvents also known as modifiers. A material becomes a critical fluid at conditions which equal its critical temperature and critical pressure. A material becomes a supercritical fluid at conditions which equal or exceed both its critical temperature and critical pressure. The parameters of critical temperature and critical pressure are intrinsic thermodynamic properties of all sufficiently stable pure compounds and mixtures. Carbon dioxide, for example, becomes a supercritical fluid at conditions which equal or exceed its critical temperature of 31.1° C. and its critical pressure of 72.8 atm (1,070 psig). In the supercritical fluid region, normally gaseous substances such as carbon dioxide become dense phase fluids which have been observed to exhibit greatly enhanced solvating power. At a pressure of 3,000 psig (204 atm) and a temperature of 40° C., carbon dioxide has a density of approximately 0.8 g/cc and having a dipole moment of zero Debye, behaves much like a nonpolar organic solvent.

A supercritical fluid displays a wide spectrum of solvation power as its density is strongly dependent upon temperature and pressure. Temperature changes of tens of degrees or pressure changes by tens of atmospheres can change a compound solubility in a supercritical fluid by an order of magnitude or more. This feature allows for the fine-tuning of solvation power and the fractionation of mixed solutes. The selectivity of nonpolar supercritical fluid solvents can also be enhanced by addition of compounds known as modifiers (also referred to as entrainers or cosolvents). These modifiers are typically somewhat polar organic solvents such as acetone, ethanol, methanol, methylene chloride or ethyl acetate. Varying the proportion of modifier allows wide latitude in the variation of solvent power.

In addition to their unique solubilization characteristics, supercritical fluids possess other physicochemical properties which add to their attractiveness as solvents. They can exhibit liquid-like density yet still retain gas-like properties of high diffusivity and low viscosity. The latter increases mass transfer rates, significantly reducing processing times. Additionally, the ultra-low surface tension of supercritical fluids allows facile penetration into microporous materials, increasing extraction efficiency and overall yields.

A material at conditions that border its supercritical state will have properties that are similar to those of the substance in the supercritical state. These so-called “near-critical” fluids are also useful for the practice of this invention. For the purposes of this invention, a near-critical fluid is defined as a fluid which is (a) at a temperature between its critical temperature (Tc) and 75% of its critical temperature and at a pressure at least 75% of its critical pressure, or (b) at a pressure between its critical pressure (Pc) and 75% of its critical pressure and at a temperature at least 75% of its critical temperature. In this definition, pressure and temperature are defined on absolute scales, e.g., Kelvin and psia. A material property especially its polarity can be modified by use of small quantities of miscible polar entrainers such as an alcohol. To simplify the terminology, materials which are utilized under conditions that are supercritical, near-critical or exactly at their critical point with or without small molar concentrations of polar cosolvents will jointly be referred to as “SuperFluids™” fluids or referred to as “SFS™.”

SuperFluids™ [SFS™] can be used for the fractional extraction and manufacturing of highly purified cannabinoids.

Embodiments of the present invention are directed to methods of using supercritical fluids for isolating and manufacturing of cannabinoids for use as a therapeutic to treat pain, opioid addiction, multiple sclerosis, Parkinson's disease, nausea and emesis and other diseases.

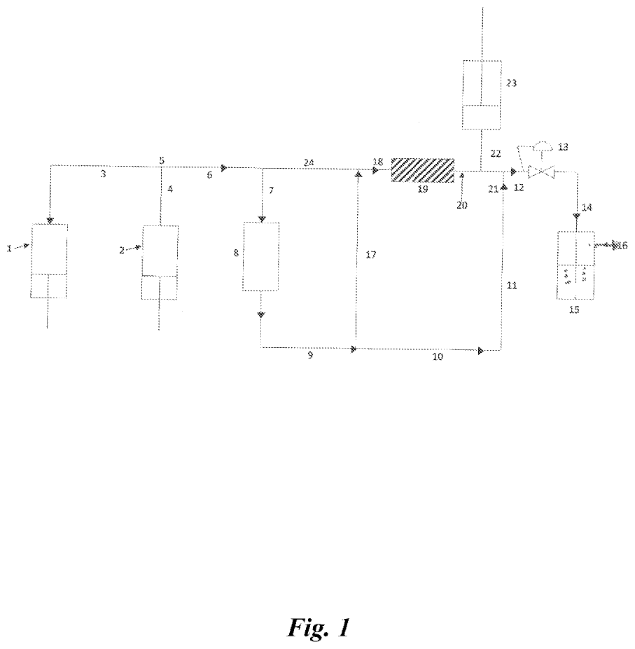

The present method and apparatus will be described with respect to FIG. 1 which depicts in schematic form the cannabinoid extraction, fractionation and purification apparatus.

Polarity-guided SuperFluids™ fractionation can be carried out on the dried and fresh Cannabis powder. SuperFluids™ CXF fractionations can be carried out on an automated extractor or a manual version of the same. As shown in FIG. 1, this system utilizes high pressure syringe or piston pump 1 for pure SFS (e.g. CO₂), high pressure or syringe pump 2 for modifier (e.g. ethanol), an extractor 8 in a cartridge holder for holding the biomass, a chromatographic column 19 for holding the chromatographic separation media, a back-pressure 13 for holding the pressure, a high pressure or syringe flush pump 23 for modifier (e.g. ethanol) to flush and keep clear the back-pressure regulator 13, and a collection vial 15.

After loading Cannabis into a cartridge on the cartridge holder 8, the fractionation procedure can start. For example, the system will be brought to 3,000 psig and 40° C., and extracted for 10 minutes with pure CO₂. The SFS CO₂ stream from 1 is routed through lines 3, 6 and 7 to an extractor 8 in a cartridge holder and then through lines 9, 10, 11 and 12 to the back-pressure regulator 13 and then via line 14 to the collection vial 15. The SFS is separated from the extractant in the collection vial and vented through line to 16, and can be recycled to SFS syringe or piston pump 1. The chromatographic column 19 is bypassed in the conduct of the fractionation procedure. Alternatively, the SFS extract from the extractor cartridge 8 can be routed via lines 9, 17 and 18 to the chromatographic column 18. During either of these processes, the back-pressure regulator 13 is continuously flushed with a small stream of flush solvent via lines 22 and 12 supplied by syringe or piston pump 23. This solvent flush clears any precipitants in the back-pressure regulator and prevents plugging. Additionally, the back-pressure regulator 13 is heated to prevent freezing during decompression due to Joule-Thompson cooling effects. This combined fraction and flush solvent is collected in a buffer or solvent such as ethanol or methanol in a collection vial, numbered 15 in FIG. 1.

The extraction parameters are then set to supercritical CO₂ at 3,000 psig and extraction temperature 40° C. for step fractionation with methanol or ethanol as cosolvent at 5, 10, 20, 30 and 40 vol % each fractionation step being 10 to mins duration. The cosolvent is introduced by high pressure or syringe pump 2 vial line 4 and mixes with the CO₂ from the high pressure or syringe pump 2 at mixing tee 5. Each biomass sample yields 6 fractions or more which are collected in ethanol or suitable solvent in separate glass vials. The fractions are then dried under vacuum in a SpeedVac, and analyzed by HPLC for cannabinoid content. Conditions which provide the highest combined content of cannabinoids are used for the base case conditions to separate and purify cannabinoids, and/or can be scaled up for manufacturing larger quantities of mixed cannabinoids.

The purification of mixed cannabinoids is initiated by routing the SFS extracts from biomass cartridge 8 that produced optimum or near-optimum mixture of cannabinoids to the chromatographic column 19 via lines 9, 17 and 18. Select cannabinoids are deposited out of the SFS stream onto the head of the chromatographic media in column 19. Other cannabinoids are eluted through the chromatographic column via lines 20, 21, 12 and 14, and back-pressure regulator 13 and are collected in collection vial 15. This causes separation between select cannabinoids and other cannabinoids, improving the purification of cannabinoids.

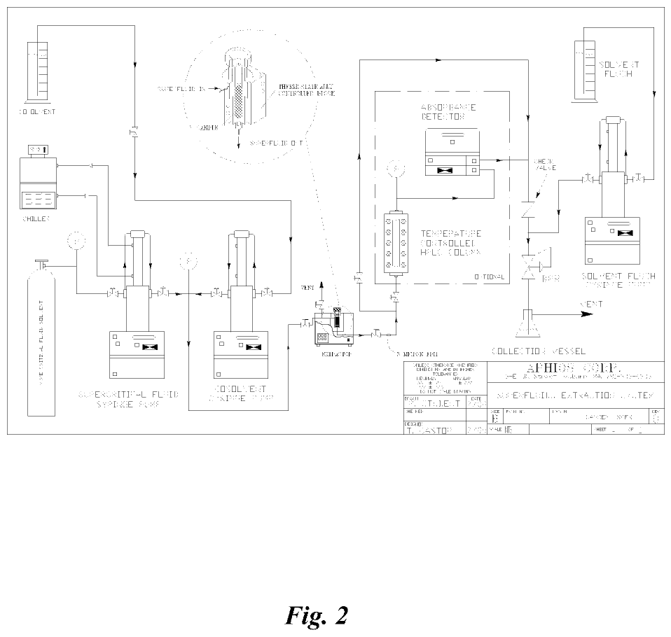

The present method and apparatus will be described with respect to FIG. 2 which depicts a process flow diagram of the SFS extraction and fractionation (SFS-CXF) apparatus.

The equipment used in FIG. 2 to perform the SFS-CXF fractionation experiments is divided into 3 major subsystems: delivery, extraction, and letdown. The delivery system consists of two Model 260-D syringe pumps (Isco, Lincoln, NE) for the supercritical fluid and the cosolvent. The two pumps are operated via a controller that can finely proportionate the delivery of the supercritical fluid and cosolvent on the basis of volume percent while maintaining delivery pressure and flow-rate, and has the capability to run programmed solvent composition gradients. The extraction system shown is an Isco Model SFX 2-10 extraction unit—a dual-well, high-pressure device that allows precise and accurate temperature control of both the sample and the supercritical fluid.

A Tescom Model 26-1722 backpressure regulator is used as the letdown system. This regulator is continuously flushed with a high pressure, low volume stream of co-solvent or methanol to displace any solids that may have deposited out of the SuperFluids™ (SFS) stream at the point of decompression. This flush also provides a continuous low-grade heat input that is sufficient to prevent freezing of the back-pressure regulator due to Joule-Thompson cooling resulting from SFS decompression. The exiting SFS stream containing extracted materials was bubbled through the cosolvent used in the extraction or methanol. The disengaged SFS is vented.

Several experiments with different SFS at different pressures and temperatures are conducted on unheated and heat-treated Cannabis sativa. SFS extraction of unheated Cannabis sativa over a range of pressure at 25° C. indicates that isolation of CBD, Δ9-THC, CBDA and 49-THCA is near optimum at 4,000 psig for near-critical, pure CO₂ at 25° C. Experiments on the effect of temperature at 5,000 psig indicated that the best extraction condition appears to be around 35° C.; the yields and absolute purities were, however, not as good as those obtained at 4,000 psig for near-critical, pure CO₂ at 25° C.

The effect of different SFS (CO₂, C₃H₈, Freon-22 and Freon-23) on the extraction of untreated Cannabis sativa was evaluated for a pressure of 5,000 psig and a temperature of 25° C.; at these conditions, near-critical propane (C₃H₈) provides the highest yields which are, however, not as good as those obtained at 4,000 psig for near-critical CO₂ at 25° C., conditions predicted by the thermodynamic analysis. The impact of different methanol co-solvent compositions on CO₂ indicated that neat CO2 resulted in the highest yields of cannabinoids. Experiments, conducted as a function of pressure for heat-treated Cannabis sativa with CO₂ at 25° C., resulted in a 49-THC yield of 68% and an absolute purity of 59%. As expected for the heat-treated Cannabis biomass, no CBDA or 49-THCA was extracted since they had been decarboxylated to CBD and A9-THC.

Based on our prior data on the use of SFS for the extraction of 49-THC and other cannabinoids from Cannabis, carbon dioxide, which has a very modest critical point (31° C. and 1,070 psia) is an excellent SFS candidate for extraction and purification of cannabinoids since it is inexpensive, non-toxic, non-flammable, and environmentally acceptable. Supercritical carbon dioxide has a density of 0.74 gm/cc at a pressure of 2,000 psia and a temperature of 40° C. At and around these conditions, CO2 behaves like an organic solvent with solvation characteristics of a liquid and the permeabilization characteristics of a gas. CO2 has a dipole moment of 0.0 Debye and behaves like the very nonpolar solvent hexane.

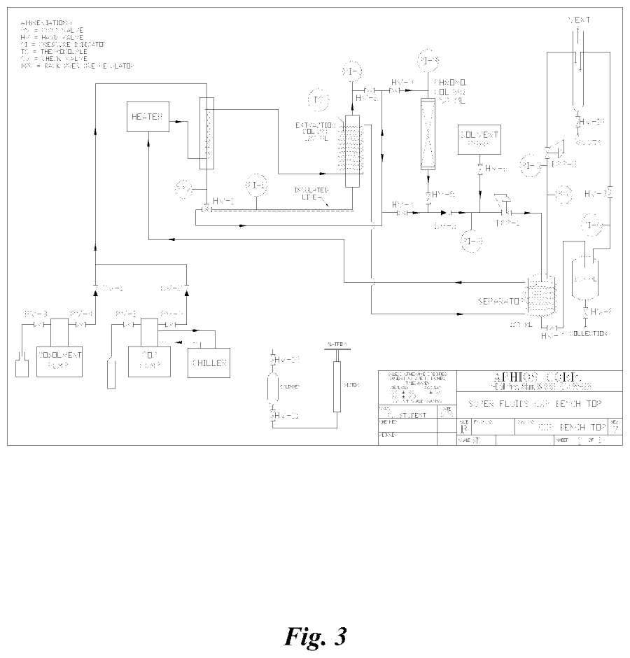

The present method and apparatus will be described with respect to FIG. 3 which depicts a process flow diagram of a bench-top apparatus for the SFS extraction and chromatographic purification (SFS-CXP) apparatus.

The apparatus utilized for the bench-top SuperFluids™ extraction and chromatography for Cannabis sativa is shown as FIG. 3. This apparatus consists of a 127 mL high-pressure extraction column and a 50 mL high-pressure chromatographic column with accessory devices for controlling temperature, pressure and flowrate of near-critical, critical and supercritical fluids with or without polar cosolvents. The entire apparatus is rated for 5,000 psig and 100° C. with operating flowrates of up to 100 mL/min. Each experiment conducted utilized around 40 grams of marijuana, a scale-up factor of ˜16 from the laboratory-scale experiments conducted in the apparatus shown as FIG. 2.

Heat-treated Cannabis (100° C. for 2.5 hours) will be first extracted in the bench top SFS-CXP apparatus to evaluate the scale-up from the laboratory-scale extraction apparatus. In a prior experiment, MAJB-1, 41.35 g of heat-treated Cannabis was extracted with near-critical CO2 at 4,000 psig and 25° C. with a flow-rate of 40 ml/min for 1.6 hours. Three fractions were taken, one every 30 minutes. The overall yield of the 49-THC target was 90.3% and the absolute purities of Δ9-THC in the three major fractions were 55.6, 68.9 and 71.1%, or an average of 65.2%.

In subsequent experiments, a silica chromatographic column was put on-line during the extraction of the heat-treated marijuana with neat CO₂. In experiment MAJB-4, 44.04 g of heat-treated marijuana was extracted with near-critical CO₂ at 4,000 psig and 25° C. at a flow-rate of 40 mL/min for 1.4 hours. The SFS extract was continuously loaded onto an activated silica chromatographic column (50 mL, 2.5 cm ID×10 cm long, 25 g). The flow through fractions were collected, dried, weighed and analyzed by HPLC. After extraction, the extraction column was bypassed and the chromatographic column eluted with a methanol:CO₂ gradient. The gradient started with 99.8% CO₂0.2% methanol for 15 minutes at a combined flowrate of 10 mL/min (˜1 extractor volume), and was increased in 0.2% increments of methanol until its concentration reached 2% in 98% CO₂. The flow through fractions were collected and analyzed by HPLC. The SFS-CXP silica chromatography clearly separated Δ9-THC from Δ9-THCA and other cannabinoids. Similar separations were obtained with CBD-enriched Cannabis.

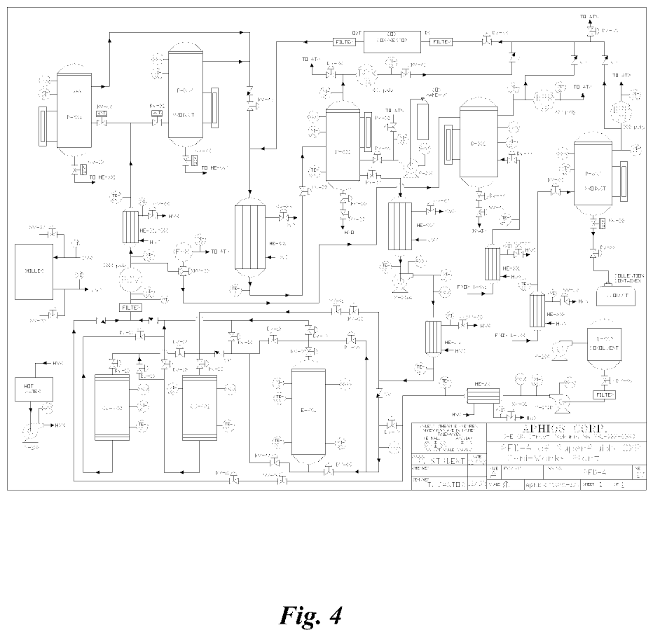

The present method and apparatus will be described with respect to FIG. 4 which depicts a process flow diagram of a semi-works plant for the SFS extraction and chromatographic purification (SFS-CXP) apparatus.

The semi-works SFS-CXP unit consists of a high-pressure 125 liters 316-SS (stainless steel) extractor, E-201, that is 5′ high with an ID of 12.5″, and two (2) 25 liters high-pressure 316-SS chromatographic columns, CC-401 and CC-402, that are each 12.5″ high with an ID of 12.5″. Both the extractor and chromatographic columns are fitted with 10-micron stainless steel filters in their end-caps that have radial distribution rings for flow uniformity. End-caps on both the extractor and chromatographic columns are a patented, quick removal design with a special safety feature that prevents their removal if there is any residual pressure in the vessels. The vessels have an ASME (American Society of Mechanical Engineers) stamp and rating for an operating pressure of 3,000 psig and temperature of 100° C. The vessels and the CXP unit are routinely tested at hydrostatic pressures of 4,000 psig.

The SFS-CXP unit is piped to allow several flow paths including: (1) extraction only with near-critical, critical or supercritical fluid with or without a co-solvent: (2) extraction with chromatography through one or both chromatographic columns; (3) chromatography only through one or both chromatographic columns.

The SFS-CXP operational runs are designed to produce standardized CB products, and extract and purify CBD, CBDA, Δ9-THC, Δ9-THCA and other cannabinoids from heat-treated and ground Cannabis biomass. Based on prior data, the CBD run is conducted with near-critical CO2 at 1,000 psig and 25° C., even though most of the bench top experiments were conducted at 4,000 psig and 25° C. The latter is conducted in an effort to isolate significant quantities of both CBD and CBDA. The data, however, suggests that it would be more efficient to conduct separate runs for CBD and CBDA. The CBD-rich CO₂ extract at 1,000 psig and 25° C. is routed through one or two chromatographic columns loaded with activated silica until the overall yield is >90%. The extractor is then bypassed and the mobile phase changed to a step gradient of methanol in CO₂ starting at 0.2% methanol:99.8% CO₂ and ending at 2% methanol:98.0% CO₂. The product is recovered in several fractions from vessels D-601 and D-602.

Additional experiments are conducted on the bench-top CXP unit, which is a 1,000 X scale-down of the semi-works CXP unit, to confirm this design and to further specify extraction times and flow-rates, and chromatographic elution gradients and flow-rates. Experiments are also conducted on the bench-top CXP unit to establish design conditions for activating and cleaning the silica in-place between runs.

While this invention has been particularly shown and described with references to specific embodiments, figures and detailed description, it will be understood by those skilled in the art that various changes in form and details may be made therein without departing from the spirit and scope of the invention as defined by the following examples.

### EXAMPLES

### Example 1: Cannabiniod Standards, HPLC Analysis and Standard Curves

Cannabinoid Standards: Four (4) standards were purchased for chromatographic assay from Alletch and ChromaDex, Santa Ana, CA. They were all purchased at certified concentrations of 1 mg/mL in methanol and transported at ambient atmosphere in sealed glass vials. The standards are as follows:
1. Cannabidiol (CBD), C₂₁H₃₀O₂, MW=314.47 g/mol, (99.9%) [Alltech]
2. Δ8-THC, C₂₁H₃₀O₂, MW=314.45 g/mol, (90.0%) [Alltech]
3. Cannabinol, C₂₁H₂₆O₂ (CBN), MW=310.42 g/mol, (98.9%) [Alltech]
4. Delta-9-Tetrahydrocannabinol (Δ9-THC), C₂₁H₃₀O₂, MW=314.45 g/mol, (97%) [ChromaDex, Santa Ana, CA]

Under Aphios' DEA Schedule I license, we requested and obtained 5 mL of 50 mg/mL Δ9-THC in absolute (100%) ethanol for use as an analytical standard in our Phase I SBIR research protocol on Dec. 27, 2002. We also requested and obtained 5 mg of impure Δ9-THCA (Lot No. JMCross 12-6-3) from the University of Mississippi on Apr. 18, 2003. This request was made to use Δ9-THCA as a standard as our research evolved to include the isolation of the carboxylic acid of Δ9-THC.

In later research, we purchased four (4) new standards for chromatographic assay from Restek Corporation, Bellefonte, PA. They were all purchased at certified concentrations of 1 mg/mL in methanol and transported on ice in sealed glass vials. The standards are as follows:
5. Cannabidiol (CBD), C₂₁H₃₀O₂, MW=314.47 g/mol, (99%) [Restek No. 34011, Lot No. A0103078]
6. Cannabinol (CBN), C₂₁H₂₆O₂, MW =310.42 g/mol, (99%) [Restek No. 34010, Lot No. A0106034]
7. Delta-9-Tetrahydrocannabinol (Δ9-THC), C₂₁H₃₀O₂, MW=314.47 g/mol, (99%) [Restek No. 34067, Lot No. A0107164]
8. Delta-9-Tetrahydrocannabinolic acid (Δ9-THCA), C₂₂H₃₀O₄, MW=358.47 g/mol, (99%) [Restek No. 34093, Lot No. A0106555]

HPLC Analysis: Two (2) HPLC methods were developed for the analysis of Δ9-THC, A-8-THC, CBN, CBD and Δ9-THCA. Since Δ9-THCA is the precursor of Δ9-THC via decarboxylation (heat) and CBN is the degradation (oxidative) product of Δ9-THC, both compounds must be resolved by the chromatography system. CBD is not psychotomimetic in pure form although it does have sedative, analgesic, and antibiotic properties. CBD can contribute to the psychotropic effect by interacting with Δ9-THC to potentiate (enhance) or antagonize (interfere or lessen) certain qualities of this effect. Δ9-THC is the main psychotomimetic (mind-bending) compound of Cannabis. Δ-8-THC is slightly less active and is reported in low concentrations, less than 1% of Δ9-THC, and may be an artifact of the extraction/analysis process.

The two HPLC methods developed were: (1) a gradient system utilizing a modified Phenomenex method; and (2) an isocratic system that is a modification of the Maripharm, Rotterdam, Netherlands method. The latter system was selected based on peak separation and product purities. This isocratic method utilized a Phenomenex Luna 3 μm C18 column (5 cm×4.6 mm) with a pre-column at 25° C. The mobile phase, at 1.0 mL/min, consisted of 78% methanol:22% water containing 1% acetic acid. Absorbance was monitored by a Waters Photodiode Array (PDA) detector, Model 996, and measured at 285 nm and 230 nm.

The analytical HPLC system included a Waters 717 Autosampler, 600E System Controller and a Waters Dual-Piston High Pressure HPLC pump, Model No. 600, driven by a Pentium 4 Personal Computer and controlled by a Waters Millenium 4.0 software. Temperature of the HPLC column was controlled by an Eppendorf CH-30 column heater. This isocratic system was utilized to analyze the Cannabis biomass and experiments MAJ-1 to MAJ-22. In order to reduce run time for Phenomenex Luna 5 and 10 μm C18 columns, the mobile phase was changed to 80% acetonitrile:20% water containing 0.1% acetic acid at a flowrate or 2.0 mL/min and a column temperature of 30° C. with absorbance measurement at 285 nm. This isocratic system was utilized to analyze fractions from experiments MAJB-1 to MAJB-10. Also, using this isocratic system, a second HPLC system (ISCO) was utilized to monitor the column chromatography utilized in the downstream purification.

A new analytical system was utilized to develop new standard curves and analyze biomass and fractions. The new analytical system consisted of a Waters 2695 Alliance Separations Module with Waters 996 Photodiode Array Detector controlled by Empower Pro software [Aphios' cGMP material code for this equipment is APH-EQ-07120]. The Alliance HPLC system is operated following Aphios' SOP No. EQ-015.

We evaluated a third HPLC method developed by Restek Corporation for their Cannabinoid-specific HPLC column, Raptor ARC-18 (Restek No. 9314A65). The Raptor ARC-18 is a 2.7 μm, 150×4.6 mm column. This HPLC method is a gradient method that included mobile phase A (0.1% formic acid in water) and a mobile phase B (0.1% formic acid in acetonitrile) with the following gradient: 25% A::75% B from 0 to 4.0 min, 0% A:100% B from 4.0 to 4.01 min and 25% A:75% B from 4.01 to 7.0 min. The gradient was run at a combined flowrate of 1.5 mL/min, the column was held at 50° C. and detection was measured at 220 nm.

Utilizing the HPLC method suggested by Restek for analyzing cannabinoids, all of the standards eluted out very close to the injection peak, CBD at 1.3 mins, CBD at 1.4 mins, THC at 1.5 mins and THCA at 1.7 mins.

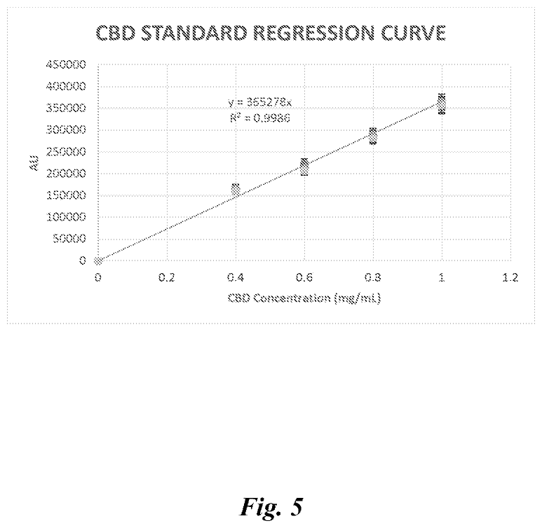

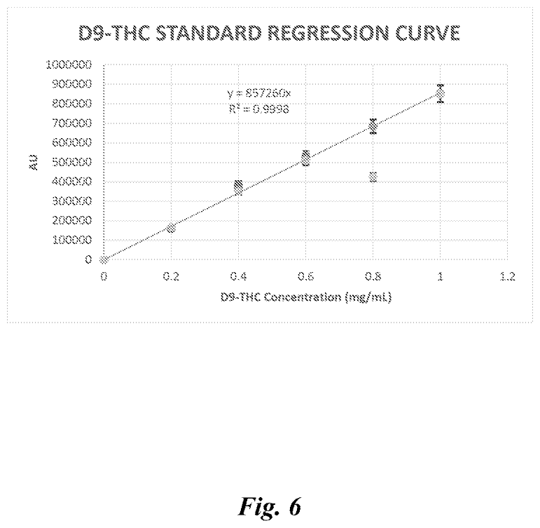

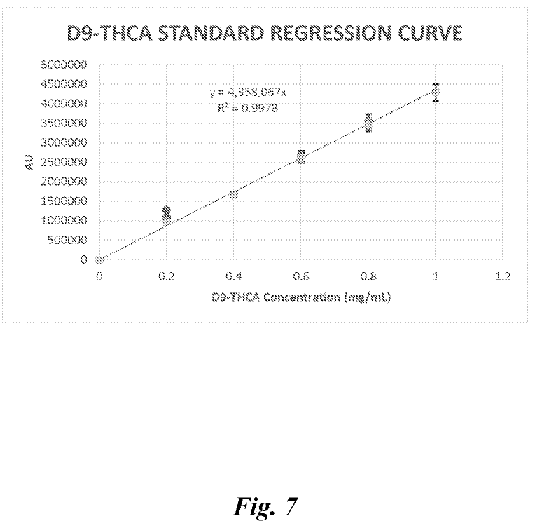

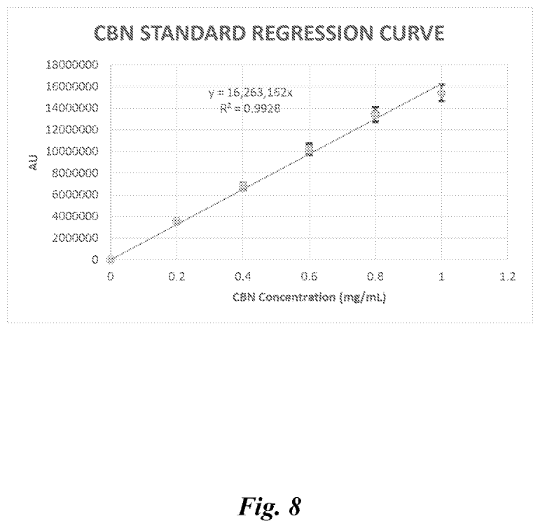

We elected to work with the modified isocratic HPLC method developed by Aphios for C18 columns. We utilized Phenomenex Luna 5 10 um C18 column, an isocratic mobile phase of 80% acetonitrile::20% water containing 0.1% acetic acid at a flowrate or 2.0 mL/min and a column temperature of 30° C. with absorbance measurement at 285 nm. The standard regressions curves for CBD, Δ9-THC, Δ9-THCA and CBN are respectively shown in FIGS. 5, 6, 7 and 8; three sample sets of the standards and their dilutions in methanol were run for each curve.

### Example 2: Extraction and Analysis of Cannabis Biomass

0.5 g of grounded Cannabis biomass was extracted four times with 20 mL of methanol:methylene chloride::90:10 by sonication. The extracts were filtered and combined in a 100 mL Class A volumetric flask. The biomass residue was then rinsed with 10 mL of the extraction solvent and added to the flask. The flask was adjusted to volume with the extraction solvent. This extract was assayed on HPLC using the isocratic system and total solids were determined. The procedure was conducted for unheated biomass and for Cannabis biomass that was oven dried@100° C. for 2.5 hours. 0.5 mL of both extracts were transferred into 1.0 mL reaction vials. The vials were then heated for 2.5 hours@100° C. using a heating block. The heated extracts were assayed on HPLC using the isocratic system. The extracted biomass was air dried under the hood and returned to the safe for inventory/usage tracking. These results are summarized in Table 1.

TABLE 1
Cannabinoid Content of Heated and Unheated
Cannabis sativa and its Extracts

| | Unheated Cannabis Biomass | | Heated Cannabis Biomass | |
|---|---|---|---|---|
| | Unheated | Heated | Unheated | Heated |
| | Extract | Extract | Extract | Extract |
| Cannabinoid | Cannabinoid Concentration (%) | | | |
| Δ9-THC | 0.93 | 8.44 | 8.43 | 8.47 |
| Δ8-THC | 0.00 | 0.00 | 0.00 | 0.00 |
| Cannabidiol | 0.15 | 0.87 | 0.87 | 0.67 |
| Cannabinol | 0.09 | 0.33 | 0.33 | 0.35 |
| Δ9-THCA | 10.7* | 0.17 | 0.17 | 0.09 |

*Estimated based on stoichiometric conversion to Δ9-THC since no standard was available at this time

### Example 3: Thermodynamic Computations

Thermodynamic calculations of the solubility of CBD, CBDA, Δ9-THC and Δ9-THCA in supercritical fluids using regular solution theory were conducted in order to guide the experimental design and analysis. For binary mixtures, the activity coefficients y1 and y2 can be calculated by using the following formulas assuming binary contributions are negligible:

$$RT ln {γ}_{1} = {V}_{1}^{L} {{Φ}_{2}^{2} ( {δ}_{1} - {δ}_{2} )}^{2} \qquad ( 1 )$$

$$RT ln {γ}_{2} = {V}_{2}^{L} {{Φ}_{1}^{2} ( {δ}_{1} - {δ}_{2} )}^{2} \qquad ( 2 )$$

The solubility parameter, δᵢ, is defined as

$${δ}_{i} = {( \frac{Δ {U}_{i}}{{V}_{i}^{L}} )}^{\frac{1}{2}} \qquad ( 3 )$$

where ΔUᵢ is the energy required to evaporate liquid i from a saturated liquid to an ideal gas, and Vᵢ^{L} is the liquid molar volume. The maximum solubility occurs when the excess Gibbs free energy is at a minimum. The excess Gibbs free energy is related to the activity coefficient by a generalization of the Gibbs-Duhem equation, given by:

$$RT ln {γ}_{i} = {( \frac{\partial {G}^{E}}{\partial {n}_{i}} )}_{T , P , {n}_{j}} \qquad ( 4 )$$

From equation (4), the minimum excess Gibbs free energy occurs when the activity coefficient is equal to one. From equations (1) and (2), the activity coefficient is only one when δ₁ is equal to δ₂. Therefore, the best conditions for supercritical extraction are predictable by calculating the Hildebrand solubility parameters of selected cannabinoids, and the supercritical fluids. For supercritical and other dense phase fluids, the Hildebrand solubility parameter can be estimated by using an empirical equation:

$$δ = 0.47 {P}_{C}^{\frac{1}{2}} ρ \qquad ( 5 )$$

The density of the supercritical fluid can be calculated by using the Hankinson-Brobst-Thomson equation. By altering system parameters such as operating temperature and pressure, the supercritical fluid's solubility parameter can be adjusted to match the solubility parameters of cannabinoids, allowing for maximum selectivity during the supercritical fluid extraction process.

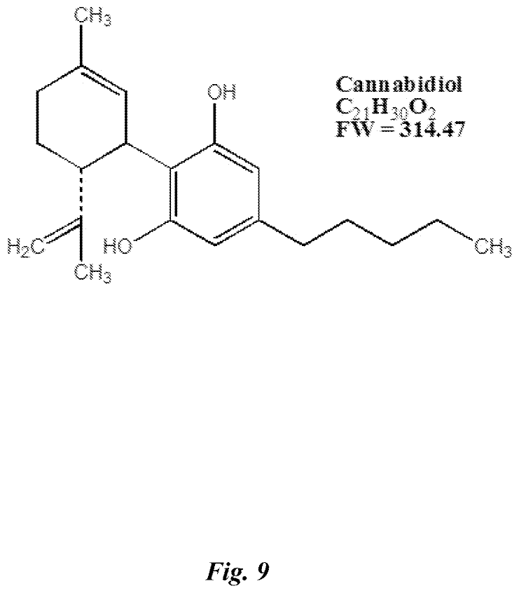

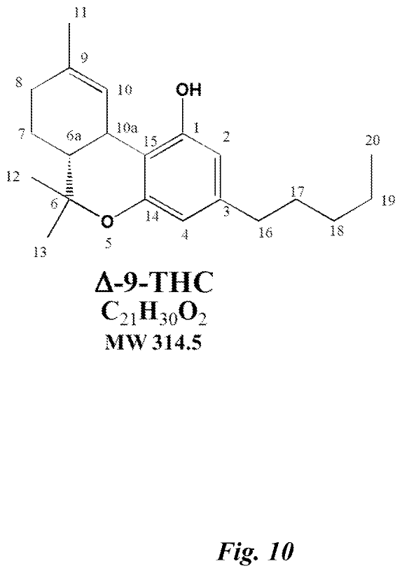

The molecular weights of CBD and Δ9-THC are identical and their structures are similar as shown in FIGS. 9 and 10. The middle ring of Δ9-THC is closed while the middle ring of CBD is open, making it slightly more polar than Δ9-THC. At room temperature, both products are oils and liable to oxygen, heat and potentially light degradation.

TABLE 2
Solubility Parameter Calculations for Δ9-THC

| Ring C# | Group | E [(J − cm3)½/mole] |
|---|---|---|
| C1 | ——C═ aromatic | 200 |
| C1 | OH aromatic | 350 |
| C2 | —CH═ aromatic | 239 |
| C3 | ——C═ aromatic | 200 |
| C4 | —CH═ aromatic | 239 |
| C14 | —C═ aromatic | 200 |
| C14 | —O(5)-ether | 235 |
| C15 | ——C═ aromatic | 200 |
| C10a | —CH— | 176 |
| C10 | —CH═ | 249 |
| C9 | ——C═ | 173 |
| C11 | —CH3 | 303 |
| C8 | —CH2— | 269 |
| C7 | —CH2— | 269 |
| C6 | ——C—— | 65 |
| C6 | —O(5)-ether | 235 |
| C6a | ——CH— | 176 |
| C13 | —CH3 | 303 |
| C12 | —CH3 | 303 |
| C16 | —CH2— | 269 |
| C17 | —CH2— | 269 |
| C18 | —CH2— | 269 |
| C19 | —CH2— | 269 |
| C20 | —CH2— | 269 |
| | | 5729 |
| A | Six-membered ring | −48 |
| B | Six-membered ring | −48 |
| C | Six-membered ring | −48 |
| A | trans (1-trans-delta) | −28 |
| A | meta - substitution | −13 |
| | | −185 |
| Δ9 - THC | TOTAL | 5544 |

Using a group contribution method as outlined by Billmeyer (1986), the solubility parameters for CBD, A9-THC, CBDA and A9-THCA were estimated as shown in Table 2 for Δ9-THC.

Based on group contribution factors, the solubility parameters [δ=(E×ρ)/MW] for CBD, Δ9-THC, CBDA and Δ9-THCA were estimated to be respectively 18.90 (J/cm³)^{1/2}, 17.63 (J/cm³)^{1/2}, 17.96 (J/cm³)^{1/2}, and 16.84 (J/cm³)^{1/2} assuming densities (ρ) of 1.0 g/cm³.

While the solubility parameter of a solid is considered to be relative stable, the solubility parameters of fluids can change drastically depending on temperature and pressure. As a fluid approaches the supercritical state, it begins to possess unique properties such as gas-like diffusivity and liquid-like density. This allows for high mass transfer and penetration rates, which facilitate the dissolution of compounds within these fluids. Due to these properties, the solubility parameters of supercritical or near critical fluids must be calculated from equations. The derived equation of state is:

$$δ ( {MPa}^{0.5} ) = {{P}_{c}^{0.5 *} [ 1 + m ( 1 - {T}_{r}^{0.5} ) ]}^{*} {c}^{0.5 *} {ρ}_{r}^{*} {z}_{c}^{- 1 *} {T}_{r}^{- 0.25}$$

where P_{c} is critical pressure, Tᵣ is reduced temperature, ρᵣ is reduced density, z_{c} is the critical compressibility factor, c is constant equal to 0.42748, and m is quadratic equation of the accentric factor described by:

$$m = 0.48 + 1.574 ω - 0.176 {ω}^{2}$$

This solubility equation can be used for non-conformal fluids, which are fluids with an accentric factor >0.1. If the fluid is conformal (ω<0.1), then the following equation can be used:

$$δ ( {MPa}^{0.5} ) = {1.132}^{*} {{P}_{c} ( MPa )}^{0.5 *} {T}_{r}^{0.25 *} {ρ}_{r}^{*} {z}_{c}^{- 0.5}$$

where P_{c} is critical pressure in MPa, Tᵣ is reduced temperature, ρᵣ is reduced density, and Z_{c} is the critical compressibility factor. In both of these cases, van der Waal's mixing rules were applied to any mixtures.
   Thermodynamic properties of selected SuperFluids are listed in Table 3.

TABLE 3
Thermodynamic Properties of Selected SuperFluids ™

| | | | Critical | Critical | Critical | Dipole |
|---|---|---|---|---|---|---|
| | Chemical | Molecular | Temperature | Pressure | Density | Moment |
| Generic Name | Formula | Weight | T_{c} (° C.) | P_{c} (psia) | (g/cc) | (Debyes) |
| Water | H₂O | 18.0 | 374.1 | 3,197.9 | 0.32 | 1.8 |
| Nitrogen | N₂ | 28.0 | −147.0 | 492.3 | 0.32 | 0.0 |
| Carbon Dioxide | CO₂ | 44.0 | 31.0 | 1,070.0 | 0.47 | 0.0 |
| Nitrous Oxide | N₂O | 44.0 | 36.4 | 1,050.8 | 0.45 | 0.2 |
| Propane | C₃H₈ | 44.1 | 96.6 | 615.8 | 0.22 | 0.084 |
| Ethylene | C₂H₄ | 28.0 | 9.2 | 730.4 | 0.22 | 0.0 |
| Freon-22 | CHClF₂ | 86.5 | 96.0 | 721.6 | 0.52 | 1.4 |
| Freon-23 | CHF₃ | 70.01 | 25.9 | 701.4 | 0.52 | 1.6 |
| HCFC-123 | CF₃CHCl₂ | 152.9 | 183.7 | 532.0 | 0.55 | 1.36 |
| HCFC-124 | CHClFCF₃ | 136.5 | 122.2 | 524.5 | 0.56 | 1.47 |
| HCFC-134a | CH₂FCF₃ | 102.0 | 101.1 | 588.9 | 0.52 | 2.06 |

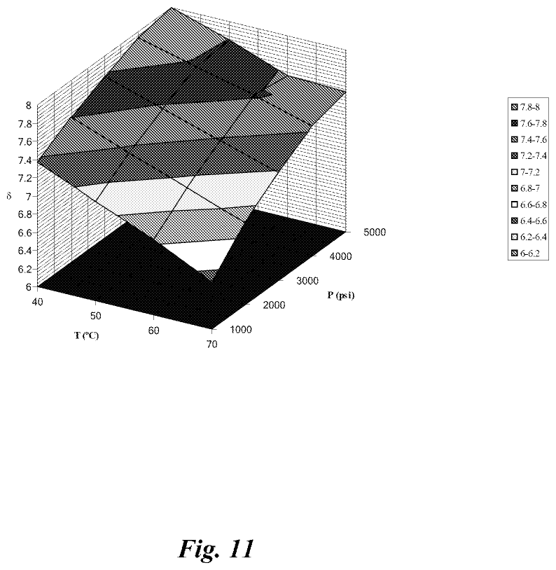

The Hildebrand Solubility parameter, δ, of SuperFluids™ as a function of temperature and pressure is shown in FIG. 11. The values listed in FIG. 11 are in (cal/cm³)^{1/2} and the conversion between (cal/cm³)^{1/2} and (J/cm³)^{1/2} is 2.045.

We developed a Hildebrand parameter calculator in Excel sheet that utilizes both the Soave-Redlich-Kwong (SRK-EOS) and Peng-Robinson (PR-EOS) Equations of State to calculate Gibbs Free Energy, Heat of Vaporization, Excess Enthalpy of Mixing and Hildebrand Solubility Parameter. The calculator was used to compute values for carbon dioxide at 5,000 psig and 25° C., 17.62 (J/cm³)^{1/2}, carbon dioxide at 4,000 psig and 25° C. to be 17.09 (J/cm³)1/2, and Freon-22 (difluorochloromethane) at 5,000 psig and 25° C. to be 16.81 (J/cm³)^{1/} to guide the initial SuperFluids™ fractionation experiments.

### Example 3: Superfluids™ Fractionation of Untreated Cannabis sativa Biomass

To prepare a sample, the biomass is first dried in a convective oven at 45° C. for 18 hours and then ground into a fine powder (approximately 40 mesh). The dried powder was transferred to a 10 mL ISCO stainless-steel extraction cartridge, after which the cartridge was sealed. After loading a cartridge on the cartridge holder, the biomass was extracted at pre-specified conditions of temperature and pressure for 1½ hours with fractions being taken every 30 minutes. After completion of the run, the spent biomass was recovered and returned to inventory control in a locked safe. The extracts were dissolved in methanol and were bought to a known volume with methanol. A portion of this was assayed by HPLC and a second portion was evaporated to dryness for determination of the solids content.

A total of 17 experiments with different SuperFluids™ at different pressures and temperatures were conducted on the first two batches of unheated Cannabis sativa, yielding 52 samples for HPLC analyses. Most of the extractions were conducted with neat supercritical or near-critical fluids without a cosolvent. One experiment was conducted with a sequential extraction/fractionation procedure in which the cosolvent methanol concentration was increased from 0% in neat supercritical CO₂ at 3,000 psig and 40° C. to 40% methanol in 5% to 10% increments, each step being 30 min at a combined flow rate of 2.0 mL/min. This experiment yielded 5 fractions, which were transferred to the separate pre-weighed glass vials.

Impact of Pressure: Data in Table 4 for the SuperFluids™ fractionation of unheated Cannabis sativa indicates that isolation of 66 9-THC and Δ9-THCA is near optimum at 4,000 psig for near-critical CO₂ at 25° C. Yield is based on the percentage of Δ9-THC and Δ9-THC recovered in the biomass relative to the analysis listed in Table 1 (10.7% Δ9-THCA and 0.93% Δ9-THC). Absolute purity of Δ9-THC is based on the standard obtained from Chromodex, Santa Ana, CA; and absolute purity of Δ9-THCA is estimated based on its conversion to Δ9-THC. The best extraction conditions were established in MAJ-08 at a pressure of 4,000 psig and a temperature of 25° C.

TABLE 4
SuperFluids ™ Extraction of Untreated Cannabis Sativa Biomass
as a Function of Pressure at a Temperature of 25° C.

| | SuperFluids ™ Extraction Parameters | | | | Δ9-THC | | Δ9-THCA | |
|---|---|---|---|---|---|---|---|---|
| Expt. | | P | T | | Yield | Absolute | Yield | Absolute |
| No. | Fluid | (psig) | (° C.) | Cosolvent | (%) | Purity (%) | (%) | Purity (%) |
| MAJ-05 | CO₂ | 1000 | 25 | none | 66.7 | 21.49 | 13.9 | 51.55 |
| MAJ-06 | CO₂ | 2000 | 25 | none | 80.6 | 14.83 | 27.7 | 58.47 |
| MAJ-07 | CO₂ | 3000 | 25 | none | 83.9 | 10.45 | 44.2 | 62.95 |
| MAJ-08 | CO₂ | 4000 | 25 | none | 100.0 | 7.61 | 88.2 | 77.20 |
| MAJ-10 | CO₂ | 5000 | 25 | none | 57.0 | 6.23 | 50.5 | 63.93 |
| MAJ-14 | CO₂ | 5000 | 25 | none | 52.7 | 7.64 | 37.7 | 62.40 |

Based on thermodynamic computations, earlier described in Example 3, these conditions should also produce near-optimum yields and purities for CBD and CBDA.

Impact of Temperature: The effect of temperature on the SuperFluids™M extraction of untreated Cannabis sativa is shown in Table 5. At 5,000 psig, the best extraction condition appears to be around 35° C. for supercritical CO2 over the narrow temperature range examined. The yields and absolute purities of Δ9-THC and Δ9-THCA are, however, not as good as those obtained at 4,000 psig for near-critical CO₂ at 25° C.

TABLE 5
SuperFluids ™ Extraction of Untreated Cannabis Sativa
Biomass as a Function of Temperature

| | SuperFluids ™ Extraction Parameters | | | | Δ9-THC | | Δ9-THCA | |
|---|---|---|---|---|---|---|---|---|
| | | P | T | | Yield | Absolute | Yield | Absolute |
| Expt. No. | Fluid | (psig) | (° C.) | Cosolvent | (%) | Purity (%) | (%) | Purity (%) |
| MAJ-10 | CO₂ | 5000 | 25 | none | 57.0 | 6.23 | 50.5 | 63.93 |
| MAJ-14 | CO₂ | 5000 | 25 | none | 52.7 | 7.64 | 37.7 | 62.40 |
| MAJ-11 | CO₂ | 5000 | 35 | none | 67.7 | 5.76 | 62.1 | 60.98 |
| MAJ-12 | CO₂ | 5000 | 45 | none | 53.8 | 5.65 | 45.9 | 55.81 |

Impact of SuperFluids™: The effect of different SuperFluids™ on the extraction of untreated Cannabis sativa is shown in Table 6 for a pressure of 5,000 psig and a temperature of 25° C.

TABLE 6
SuperFluids ™ Extraction of Untreated Cannabis Sativa Biomass
as a Function of Different SuperFluids ™

| | SuperFluids ™ Extraction Parameters | | | | Δ9-THC | | Δ9-THCA | |
|---|---|---|---|---|---|---|---|---|
| | | P | T | | Yield | Absolute | Yield | Absolute |
| Expt. No. | Fluid | (psig) | (° C.) | Cosolvent | (%) | Purity (%) | (%) | Purity (%) |
| MAJ-14 | CO₂ | 5000 | 25 | none | 52.7 | 7.64 | 37.7 | 62.40 |
| MAJ-15 | Freon-22 | 5000 | 25 | none | 33.3 | 5.00 | 24.1 | 42.15 |
| MAJ-16 | Freon-23 | 5000 | 25 | none | 35.5 | 23.53 | 4.8 | 36.06 |
| MAJ-17 | Propane | 5000 | 25 | none | 79.6 | 6.79 | 71.3 | 70.02 |

At 5,000 psig and 25° C., near-critical propane provides the highest yields of Δ9-THC and Δ9-THCA of the fluids tested. Near-critical propane is only very slightly polar, having a dipole moment of 0.084 Debyes—a factor which may also contribute to its solvation selectivity of certain complex organic molecules. The yields and absolute purities of Δ9-THC and Δ9-THCA are, however, not as good as those obtained at 4,000 psig for near-critical CO₂ at 25° C.

Freon-22 (dichlorofluoromethane) and Freon-23 (trifluoromethane), with their large dipole moments, were tested to determine their selective extraction efficiencies relative to carbon dioxide. Freon-23 was of particular interest since it is non-chlorinated. These fluids were not as effective as CO₂ or propane and were thus removed from further consideration. Propane was also eliminated since it is much more expensive, toxic and flammable than carbon dioxide. SuperFluids™ CO₂ can be recovered and recycled, and any methanol cosolvent utilized can also be recovered and recycled, in order to minimize operating costs and environmental impact.

Impact of Cosolvent: The SuperFluids™ CO₂ fractionation of untreated Cannabis sativa biomass as a function of different cosolvent concentrations at 25° C. and 5,000 psig are summarized in Table 7. For these sub-optimal conditions for CO₂, most of the Δ9-THC and Δ9-THCA are extracted in the first fraction (0% methanol). However, an additional 20% of Δ9-THC and 25% Δ9-THCA were recovered in the second fraction (5% methanol), increasing the overall yield. Under optimal conditions for CO₂, a cosolvent will not be required for Δ9-THC as shown in Table 4. A cosolvent may, however, be useful for the recovery of Δ9-THCA and CBDA, which are slightly more polar than Δ9-THC and CBD, respectively.

TABLE 7
SuperFluids ™ CO₂ Extraction of Untreated Cannabis Sativa
Biomass as a Function of Different Cosolvent Concentrations
at Temperature of 25° C. and Pressure of 5,000 psig

| | SuperFluids ™ Extraction Parameters | | | | Δ9-THC | | Δ9-THCA | |
|---|---|---|---|---|---|---|---|---|
| | | P | T | | Yield | Absolute | Yield | Absolute |
| Expt. No. | Fluid | (psig) | (° C.) | Cosolvent | (%) | Purity (%) | (%) | Purity (%) |
| MAJ-09-01 | CO₂ | 5000 | 25 | 0% methanol | 43.0 | 5.40 | 42.3 | 61.13 |
| MAJ-09-02 | CO₂ | 5000 | 25 | 5% methanol | 8.6 | 4.28 | 10.9 | 62.46 |
| MAJ-09-03 | CO₂ | 5000 | 25 | 10% methanol | 2.2 | 2.96 | 2.2 | 44.93 |
| MAJ-09-04 | CO₂ | 5000 | 25 | 20% methanol | 2.2 | 1.94 | 2.5 | 22.24 |
| MAJ-09-05 | CO₂ | 5000 | 25 | 40% methanol | 0.00 | 1.20 | 0.3 | 6.92 |
| MAJ-09 | CO₂ | 5000 | 25 | N/A | 56.0 | 4.59 | 58.2 | 54.66 |

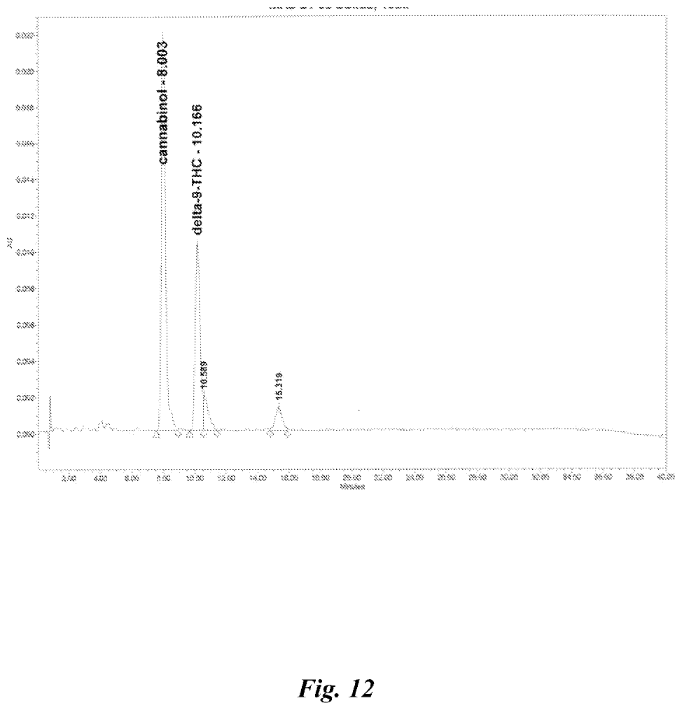

SuperFluids™ Extraction of Heated Cannabis Biomass: Six (6) SuperFluids™ extraction experiments were conducted on the first two batches of Cannabis sativa biomass that had been heated at 100° C. for 2.5 hours to convert the Δ9-THCA into Δ9-THC. The results of these experiments are listed in Table 8, in which the yield for Δ9-THC is based on the 8.43% value for the cannabinoid content of heated biomass listed in Table 1. These experiments conducted as a function or pressure with CO₂ at 25° C. indicate that the best pressure for extracting 49-THC from heat-treated Cannabis biomass is 1,000 psig, resulting in a yield of 70.3% and an absolute purity of 68.5%. Based on thermodynamic computations, these conditions will be near-optimum for the extraction of CBD from CBD-enriched Cannabis sativa biomass. An HPLC chromatogram of the primary fraction of MAJ-21 is shown as FIG. 12. As expected for the heat-treated Cannabis biomass, no Δ9-THCA was extracted in any of the experiments conducted on the heat-treated marijuana.

TABLE 8
SuperFluids ™ CO₂ Extraction of Heat-Treated Cannabis Sativa
Biomass as a Function of Pressure at a Temperature of 25° C.

| | SuperFluids ™ Extraction Parameters | | | | Δ9-THC | | Δ9-THCA | |
|---|---|---|---|---|---|---|---|---|
| | | P | T | | Yield | Absolute | Yield | Absolute |
| Expt. No. | Fluid | (psig) | (° C.) | Cosolvent | (%) | Purity (%) | (%) | Purity (%) |
| MAJ-18 | CO₂ | 2000 | 25 | none | 30.1 | 28.06 | 0.00 | 0.00 |
| MAJ-19 | CO₂ | 4000 | 25 | none | 68.1 | 59.35 | 0.00 | 0.00 |
| MAJ-20 | CO₂ | 5000 | 55 | none | 62.9 | 56.30 | 0.00 | 0.00 |
| MAJ-21 | CO₂ | 1000 | 25 | none | 70.3 | 68.51 | 0.00 | 0.00 |
| MAJ-22 | CO₂ | 3000 | 25 | none | 61.2 | 59.55 | 0.00 | 0.00 |
| MAJ-23 | CO₂ | 5000 | 25 | none | 60.4 | 56.26 | 0.00 | 0.00 |

### Example 4: Superfluids™ Extraction and Chromatography of Heat-Treated Cannabis Biomass

Heat-treated Cannabis (100° C. for 2.5 hours) was first extracted in the bench top SFS CXP apparatus (FIG. 3) to evaluate the scale-up from the laboratory-scale extraction apparatus (FIG. 2). In this experiment, MAJB-1, 41.35 g of heat-treated marijuana was extracted with near-critical CO2 at 4,000 psig and 25° C. with a flowrate of 40 mL/min for 1.6 hours. Three fractions were taken, one every 30 minutes. The overall yield of the Δ9-THC target was 90.3% and the absolute purities of Δ9-THC in the three major fractions were 55.6, 68.9 and 71.1%, averaging 65.2%. The overall yield was computed after exhaustively extracting the spent biomass with organic solvents to determine residual Δ9-THC content.

In subsequent experiments, a silica chromatographic column was put on-line during the extraction of the heat-treated marijuana with neat CO₂. In MAJB-4, 44.04 g of heat-treated marijuana was extracted with near-critical CO₂ at 4,000 psig and 25° C. at a flowrate of 40 mL/min for 1.4 hours. The SFS extract was continuously loaded onto an activated silica chromatographic column (50 mL, 2.5 cm ID×10 cm long, 25 grams). The flow through fractions are collected, dried, weighed and analyzed by HPLC. After extraction, the extraction column is bypassed and the chromatographic column is eluted with a methanol:CO₂ gradient. The gradient started with 0.2% methanol/99.8% CO₂ for 15 minutes at a combined flowrate of 10 mL/min (˜1 extractor volume), and was increased in 0.2% increments for methanol until its concentration was 2% in 98% CO₂. The flow through fractions were collected and analyzed by HPLC.

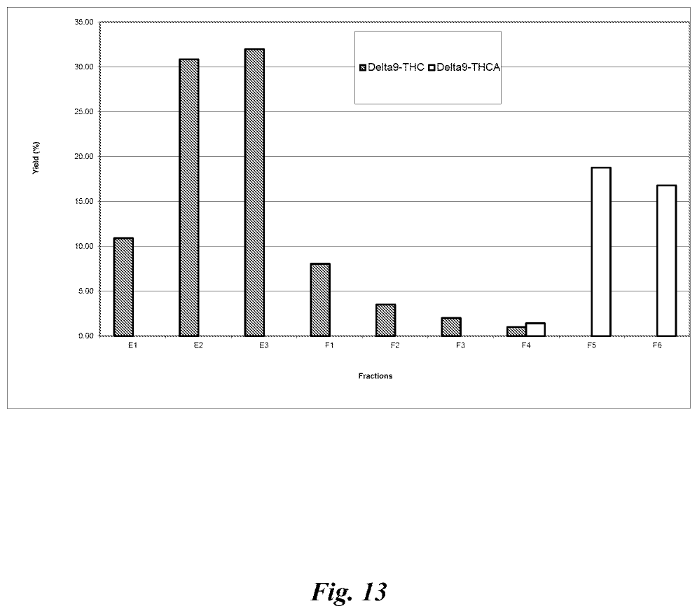

The results of MAJB-4 are shown in FIG. 13. E1, E2 and E3 are the flow through fractions obtained during the extraction step. F1 to F6 are fractions obtained during the chromatographic step; F7-F11, which were free of the target cannabinoids, are not reported. The SFS CXP silica chromatography clearly separates 49-THC from Δ9-THCA. The absolute purities of the 49-THC were 54.1, 65.0 and 68.7% for fractions E1, E2 and E3. The extraction and elution profiles in FIG. 13 show good separation between Δ9-THC and Δ9-THCA.

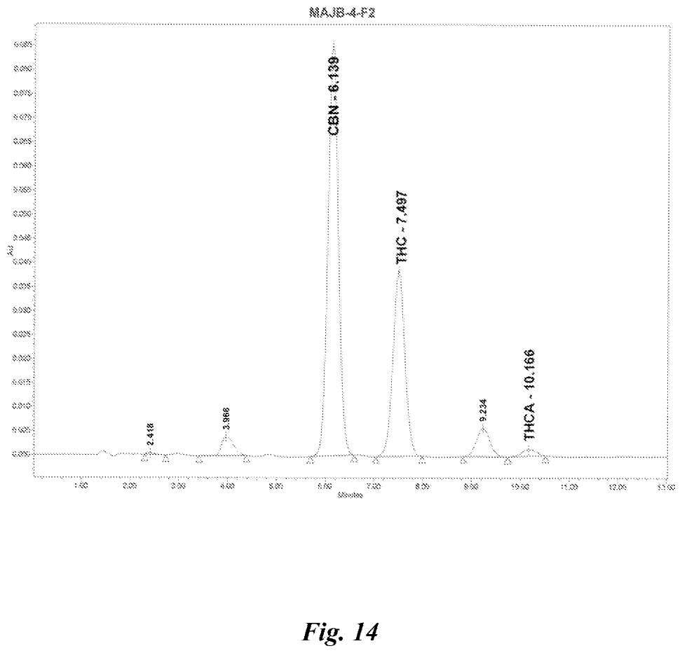

An HPLC chromatogram of the middle extraction fraction, E2, is shown as FIG. 14. In this chromatogram, the other major peak is cannabinol (CBN). The relative mass of CBN to Δ9-THC is about 15-20 time less, even though it appears much greater because of its high UV response factor. CBN is a degradation (oxidative) product of Δ9-THC and is thought to be generated by poor storage and/or processing conditions.

Several other runs were conducted to evaluate the relative amount of silica required to improve the separation efficiency between, Δ9-THC and CBN. These experiments did not improve the separation efficiency since they were all conducted with increasing ratios of silica to Cannabisbiomass. Since Δ9-THC appears to have some affinity for silica, the ratios will be decreased in further studies.

Use of C18 instead of silica in MAJB-5, all other conditions being the same as MAJB-4, worsened the separation as 49-THCA was obtained in all the flow through extraction step fractions [data not shown]. The overall recovery efficiencies, however, remained around the same (˜ 85%) as MAJB-4 since all the other run conditions were the same.

### Example 5: Superfluids™ Extraction and Chromatography of Unheated Cannabis Biomass

Several experiments on the SuperFluids™ extraction and chromatography (CXP) of untreated Cannabis biomass were also conducted on the bench top CXP apparatus (FIG. 3). These experiments were also conducted with CO₂ at 4,000 psig and 25° C. The first experiment, MAJB-2, resulted in a low overall Δ9-THCA yield of 37.4%. In the next experiment, the extraction time with neat near-critical CO₂ was doubled and the overall yield increased to 79.7%. The silica column was then eluted with gradient of 99.8% CO₂:0.2% methanol to 98% CO_{2:2}% methanol. The results of this experiment, MAJB-3, are listed in Table 9.

TABLE 9
SuperFluids ™ Extraction and Chromatography of Untreated
Cannabis Sativa Biomass At a Pressure of 4,000 psig and Temperature of 25° C.

| | Fluid | Cosolvent | | | Δ9-THCA | | |
|---|---|---|---|---|---|---|---|
| | CO₂ | Methanol | Time | Column | Amount | Yield | Abs. Purity |
| Fraction | (%) | (%) | (mins) | Volumes | (mg) | (%) | (%) |
| E1 | 100 | 0 | 28.6 | 9 | 0.0 | 0.0 | 0.0 |
| E2 | 100 | 0 | 28.6 | 9 | 0.0 | 0.0 | 0.0 |
| E3 | 100 | 0 | 28.6 | 9 | 0.0 | 0.0 | 0.0 |
| E4 | 100 | 0 | 28.6 | 9 | 35.9 | 1.39 | 33.9 |
| E5 | 100 | 0 | 28.6 | 9 | 139.4 | 5.41 | 60.1 |
| E6 | 100 | 0 | 28.6 | 9 | 196.7 | 7.64 | 74.5 |
| F1 | 99.8 | 0.2 | 15 | 3 | 74.5 | 2.89 | 58.7 |
| F2 | 99.6 | 0.4 | 15 | 3 | 108.6 | 4.22 | 68.7 |
| F3 | 99.4 | 0.6 | 15 | 3 | 99.9 | 3.88 | 78.7 |
| F4 | 99.2 | 0.8 | 15 | 3 | 100.3 | 3.89 | 78.4 |
| F5 | 99.0 | 1.0 | 15 | 3 | 183.4 | 7.12 | 78.7 |
| F6 | 98.8 | 1.2 | 15 | 3 | 280.4 | 10.89 | 64.8 |
| F7 | 98.6 | 1.4 | 15 | 3 | 411.1 | 15.96 | 77.6 |
| F8 | 98.4 | 1.6 | 15 | 3 | 343.0 | 13.32 | 83.3 |
| F9 | 98.2 | 1.8 | 15 | 3 | 75.3 | 2.92 | 40.3 |
| F10 | 98.0 | 2.0 | 15 | 3 | 5.4 | 0.21 | 7.5 |
| Totals | | | 321 | 57 | 2053.9 | 79.74 | |
| Residue | | | | | 522.0 | | |

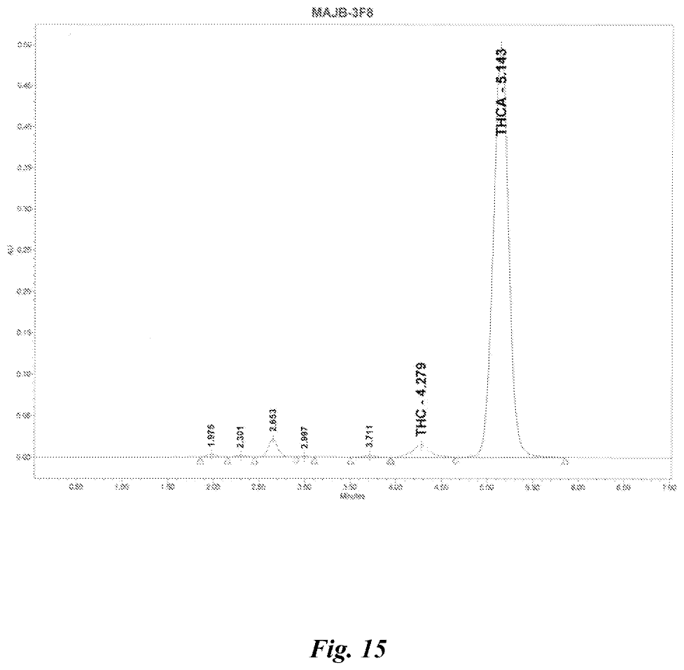

In Table 9, fractions E1-E6 were collected after flowing the neat CO₂ Cannabis extract though a silica column, as previously described. The mobile phase was then changed to a CO2:methanol gradient, the extraction column bypassed, the chromatographic column eluted at a lower flowrate and fractions F1 to F10 collected. The absolute purities of Δ9-THCA in the major fractions ranged from 59% to 83%. An HPLC chromatogram of fraction MAJB-3-F8, is shown as FIG. 15.

The overall yield of MAJB-3 could have been increased beyond 80% by either increasing the extraction time with neat CO₂ and/or by adding a cosolvent step, as suggested by the data in Table 7. The latter was demonstrated in MAJB-9 by following the neat CO₂ extractions steps with a CO₂:methanol::95:5 step. MAJB-9 resulted in an overall Δ9-THCA yield of 92.6%. In MAJB-10, the extraction step was increased by 50% over MAJB-3. The A9-THCA-rich CO2 stream was routed through the silica column and eluted as previously described. The Cannabis biomass was then extracted with 5% methanol:95% CO2.

MAJB-10 resulted in an overall 49-THCA yield of 85.4% with high absolute purities ranging from 59% to 82%. These results suggest that a combination of increased extraction with neat CO2 and use of a small amount of cosolvent (5% methanol or less) will be optimum for isolating 49-THCA.

### Example 6: Superfluids™ Extraction and Chromatographic Purification of CBD From Cannabis sativa (CBD-I-24)

Based on prior examples, exhaustive experiments were conducted on heated Cannabis sativa (90 mins at 120° C.) with CO₂ at 1,000 psig and 25° C. (CBD-I-15) and 3,000 psig and 50° C. (CBD-I-16) in the SFS-CXF fractionation unit (FIG. 2). The data indicated that CO₂ at 3,000 psig and 50° C. was more efficent in isolating CBD with a 93% yield and Δ9-THC with a 60% yield. Experiment CBD-I-16 was scaled up in the SFS-CXP bench-top unit (FIG. 3) by a factor of 16.8 which results in a 96% yield of CBD with an absolute purity of ˜70% in the CO₂ extraction step. The chromatographic column was online but became plugged because of the high amount of biomass extracted.

Unheated but dried and ground Cannabis sativa was then processed in the SFS-CXP bench top unit (FIG. 3), which is a 1:1000 scale down of Aphios' pilot SFS-CXP unit including the extractor and chromatographich columns (FIG. 4). The first 5 fractions of this experiment CBD-I-24 were obtained with CO₂ at 3,000 psig and 50° C. going into the extractor containing ˜40 g ground, dried Cannabis sativa and the chromatographich columnns containing ˜25 g heat-dried silica.

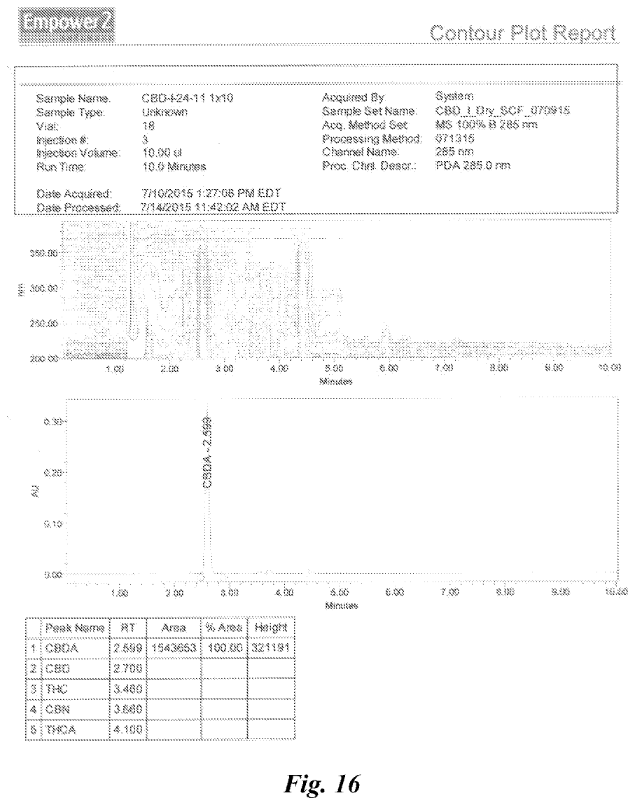

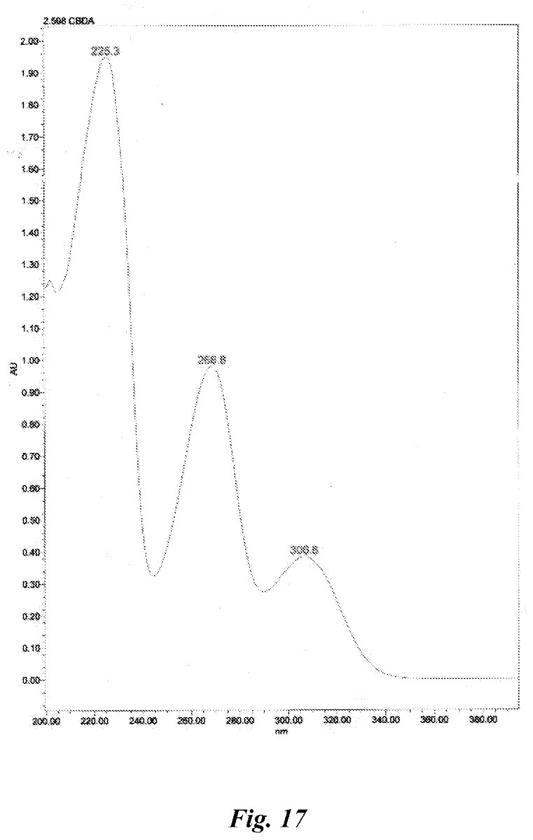

SFS chromatography with 98.5% CO₂ and 1.5% methanol pushed by 100% CO2 at 3,000 psig and 27° C. totally separated CBDA with a chromatographic purity of 100% (FIGS. 16 and 17) and 0.0% in Fraction 11 which produced ˜2 g CBDA with absolute purities from 95% to 100%.

### Example 7: Superfluids™ Extraction and Purification of CBDA from Cannabis sativa (CBD-II-38)

The run sheet of CBD-II-38, from which promising results were obtained prior to the rigorous control of process parameters and conditions, following Aphios SOP APH-S0201 Ver. 3 are shown as Table 10. The results are shown in Table 11 which highlights the yield of 1.971 g of 100% chromatographically pure CBDA in only two fractions (#11 and 12) for an overall yield of 7.40% where yield is percent recovered compared to feed, and a recovery efficiency of 110.5%.

TABLE 10
Run Sheet for SuperFluids ™ Extraction and Purification of CBDA from Untreated Cannabis Sativa (CBD-II-38)

| Experiment No. | CBD-I-38 | | | | | |
|---|---|---|---|---|---|---|
| Date of Experiment: | 26 Feb. 16 | | | | | |
| Designed by: | TC | Performed by: | TPC, KDL, WAB, RJS | | | |
| Biomass: | Cannabis sativa | Batch No: | RTI-13784-1114-18-6 | #1 | | |
| | | CB Content of Non-derivatized Extracts: | | D9-THC (%) | 4.00 | CBD (%) |
| | | CB Content of Derivatized Extracts: | | D9-THC (%) | 0.57 | CBD (%) |
| Pressure (psig): | 3000 | Preparation: | Biomass was directly packed into extraction column. | | | |
| Temperature (° C.): | 57 | | Not dried or ground. | | | |
| Total Flowrate (mL/min): | 40 | | | | | |
| BPR Flush (mL/min): | 2 | Procedure: | Aphios SOP No. APH-S0201 | | | |
| Original Biomass (g): | 26.71 | | Aphios SOP No. APH-EQ-015 | | | |
| SFS Extracted Biomass (g): | 22.5572 | | | | | |

| | | | |
|---|---|---|---|
| Biomass Extracted (mg): | 4152.8 | Equipment: | SFS-CXP Bench-Top Equipment |

| | | | | | | Extracted | | |
|---|---|---|---|---|---|---|---|---|
| | Time | CO2 | MeOH | | Vol. | Sample | *Yield | |
| CBD-II-38 | (min) | (% I) | (%) | Color | (mL) | (mg) | (%) | |
| 1 | 22 | 100 | 0 | No color | 15.00 | ND | ND | Extraction & Deposition |
| 2 | 15 | 100 | 0 | No color | 13.00 | ND | ND | Extraction & Deposition |
| 3 | 14 | 100 | 0 | No color | 17.00 | ND | ND | Extraction & Deposition |
| 4 | 22 | 100 | 0 | No color | 17.00 | ND | ND | Extraction & Deposition |
| 5 | 13 | 100 | 0 | No color | 17.00 | ND | ND | Extraction & Deposition |
| 6 | 33 | 0 | 100 | gold, grn/precip | 34.00 | 141.10 | 0.528 | BPR-1 Wash |
| 7 | 8 | 100 | 0 | yellow/precip | 30.00 | 136.05 | 0.509 | Chromatographic Elution |
| 8 | 16 | 99.5 | 0.5 | pale yell/precip | 58.00 | 103.82 | 0.389 | Chromatographic Elution |
| 9 | 16 | 99 | 1 | yellow/precip | 46.00 | 34.04 | 0.127 | Chromatographic Elution |
| 10 | 17 | 98.5 | 1.5 | pale wheat/precip | 35.00 | 63.35 | 0.237 | Chromatographic Elution |
| 11 | 17 | 98 | 2 | yellow/precip | 41.00 | 842.55 | 3.154 | Chromatographic Elution |
| 12 | 17 | 100 | 0 | amber, gold/precip | 32.00 | 752.32 | 2.817 | Chromatographic Elution |
| 13 | 16 | 100 | 0 | amber, gold/precip | 28.00 | 150.92 | 0.565 | Chromatographic Elution |
| 14 | 19 | 100 | 0 | yellow, grn/precip | 47.00 | 99.88 | 0.374 | Chromatographic Elution |
| 15 | 20 | 60 | 40 | brown, grn/precip | 110.00 | 577.50 | 2.162 | Chromatographic Elution |
| 16a | 16 | 0 | 100 | pale grn/precip | 96.00 | 80.64 | 0.302 | Chromatographic Elution |
| 16b | | 0 | 100 | pale grn/precip | 61.00 | 9.76 | 0.037 | Chromatographic Elution |
| 17a | 23 | 0 | 100 | pale grn/precip | 76.00 | 19.76 | 0.074 | Chromatographic Elution |
| 17b | | 0 | 100 | cloudy wheat | 35.00 | 5.25 | 0.020 | Chromatographic Elution |
| 18 | ND | ND | ND | pale yellow/precip | 5.00 | ND | ND | Chromatographic Elution |
| 19 | ND | ND | ND | pale yell-grn/precip | 120.00 | ND | ND | Chromatographic Elution |
| TOTAL | 304 | N/A | N/A | | 808.00 | 3,016.94 | 11.295 | |
| Spent | | 0 | 100 | | | | 0.000 | |
| Biomass | | | | | | | | |

TABLE 10
Run Results of SuperFluids ™ Extraction and Purification of CBDA from Untreated Cannabis Sativa (CBD-II-38)

| | CBDA | CBD | THC | CBN | THCA | CBDA | CBD | THC | CBN | THCA | |
|---|---|---|---|---|---|---|---|---|---|---|---|
| CBD-II-38 | (mg) | (mg) | (mg) | (mg) | (mg) | (% AP) | (% AP) | (% AP) | (% AP) | (% AP) | |
| 1 | 0.00 | 0.00 | 0.00 | 0.00 | 0.00 | ND | ND | ND | ND | ND | Extraction & Deposition |
| 2 | 0.00 | 0.00 | 0.00 | 0.00 | 0.00 | ND | ND | ND | ND | ND | Extraction & Deposition |
| 3 | 0.00 | 0.00 | 0.00 | 0.00 | 0.00 | ND | ND | ND | ND | ND | Extraction & Deposition |
| 4 | 0.00 | 0.00 | 0.00 | 0.00 | 0.00 | ND | ND | ND | ND | ND | Extraction & Deposition |
| 5 | 0.00 | 0.00 | 0.00 | 0.00 | 0.00 | ND | ND | ND | ND | ND | Extraction & Deposition |
| 6 | 0.80 | 70.12 | 20.13 | 1.63 | 0.43 | 0.56 | 49.70 | 14.27 | 1.15 | 0.31 | BPR-1 Wash |
| 7 | 0.79 | 25.96 | 59.68 | 5.68 | 6.92 | 0.58 | 19.08 | 43.87 | 4.18 | 5.08 | Chromatographic Elution |
| 8 | 0.60 | 17.23 | 43.24 | 5.22 | 10.25 | 0.57 | 16.60 | 41.65 | 5.03 | 9.87 | Chromatographic Elution |
| 9 | 0.00 | 7.85 | 6.16 | 0.97 | 0.85 | 0.00 | 23.07 | 18.10 | 2.85 | 2.49 | Chromatographic Elution |
| 10 | 22.42 | 3.76 | 13.71 | 1.15 | 0.64 | 35.38 | 5.93 | 21.64 | 1.82 | 1.01 | Chromatographic Elution |
| 11 | 1,464.76 | 0.00 | 0.00 | 0.00 | 0.00 | 173.85 | 0.00 | 0.00 | 0.00 | 0.00 | Chromatographic Elution |
| 12 | 506.26 | 0.00 | 0.00 | 0.00 | 0.00 | 67.29 | 0.00 | 0.00 | 0.00 | 0.00 | Chromatographic Elution |
| 13 | 23.70 | 5.03 | 0.75 | 0.03 | 6.92 | 15.71 | 3.33 | 0.50 | 0.02 | 4.59 | Chromatographic Elution |
| 14 | 10.13 | 0.00 | 0.00 | 0.00 | 6.04 | 10.14 | 0.00 | 0.00 | 0.00 | 6.05 | Chromatographic Elution |
| 15 | 74.46 | 0.00 | 0.00 | 0.00 | 24.29 | 12.89 | 0.00 | 0.00 | 0.00 | 4.21 | Chromatographic Elution |
| 16a | 1.18 | 0.00 | 0.00 | 0.00 | 0.00 | 1.47 | 0.00 | 0.00 | 0.00 | 0.00 | Chromatographic Elution |
| 16b | 0.43 | 0.00 | 0.00 | 0.00 | 0.00 | 4.41 | 0.00 | 0.00 | 0.00 | 0.00 | Chromatographic Elution |
| 17a | 0.00 | 0.00 | 0.00 | 0.00 | 0.00 | 0.00 | 0.00 | 0.00 | 0.00 | 0.00 | Chromatographic Elution |
| 17b | 0.07 | 0.00 | 0.00 | 0.00 | 0.00 | 1.40 | 0.00 | 0.00 | 0.00 | 0.00 | Chromatographic Elution |
| 18 | 0.61 | 0.00 | 0.04 | 0.00 | 0.00 | ND | ND | ND | ND | ND | |
| 19 | 0.95 | 0.00 | 0.00 | 0.01 | 1.17 | ND | ND | ND | ND | ND | |
| TOTAL | 2107.16 | 129.95 | 143.73 | 14.68 | 56.33 | 69.84 | 4.31 | 4.76 | 0.49 | 1.87 | |
| YIELD (%) | 7.89 | 0.49 | 0.54 | 0.05 | 0.21 | N/A | N/A | N/A | N/A | N/A | |
| Spent | 0.00 | 0.00 | 0.00 | 0.00 | 0.00 | #DIV/0! | #DIV/0! | #DIV/0! | #DIV/0! | #DIV/0! | |
| Biomass | | | | | | | | | | | |
| RECOVERY (%) | 117.75 | 7.26 | 13.45 | 19.62 | 6.39 | | | | | | |

*Yield = (Extracted Sample/Original Biomass)*100
**Recovery = (Yield/RTI Assayed Content)*100

### Example 8: Superfluids™ Extraction of CBDA from Cannabis sativa(CBD-II-45 to CBD-II-55)

Several bench-top SFS-CXP experiments were conducted to establish operating conditions to define biomass preparation such as ground or unground, undried and dried to remove moisture, and unheated or heated to convert CBDA into CBD and THCA into THC; confirm operating pressure conditions of 3,000 psig±300 psig; operating temperature, 50° C. +10° C.; CO₂ flowrate for extraction ranging from 40 mL/min to 60 ml/min; extraction time for cannabinoid extraction; and CO₂:methanol gradient, flowrates and elution times for chromatographic purification of extracted cannabinoids.

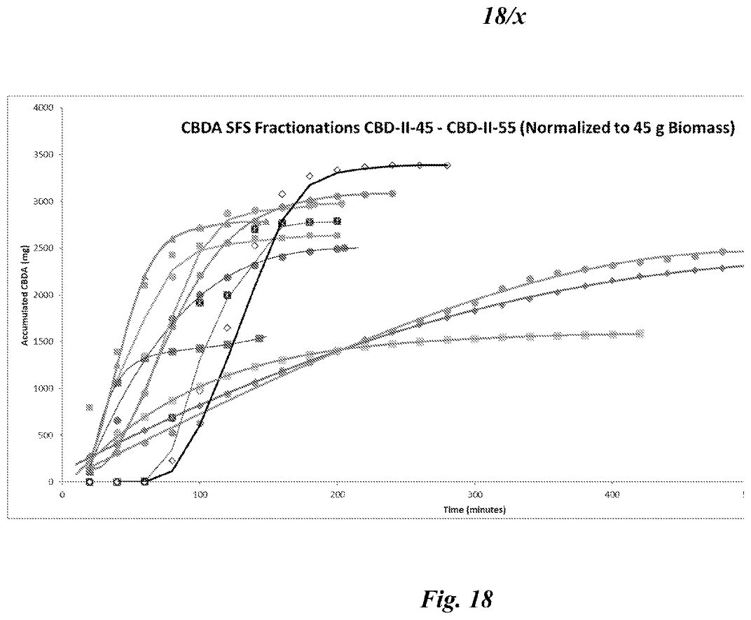

The amount of biomass loaded varied from 20 g of unground to 45 g of ground Cannabis, the maximum loading in the 127 mL extractor in the SFS-CXP bench top unit. While 20 g of unground biomass was selected as a scale-up parameter for running 20 kg in the pilot-scale unit, up to 27 g of unground biomass was packed into the extractor for some experiments. The CBDA elution profiles of these experiments are shown as a function of time in FIG. 18; these elution profiles are normalized to 45 grams. These experiments were conducted to establish the best conditions for extracting CBDA.

### Example 9: Superfluids™ Extraction and Chromatographic Purifcation of Cannabinoids from Cannabis sativa (CBD-II-57)

The run conditions for CBD-II-57 are shown in Table 11. 20.0 g marijuana biomass (unground and unheated) was extracted with SFS CO₂ at 3,000 psig and 50° C., the extract was deposited onto 23 g silica (heated at 117° C. for 15 hours) and then eluted with a step gradient of CO₂:methanol at 99.5%:0.5%, 99.0%: 1.0%, 98.5%: 1.5% and 98.0%:2.0% ratios at 3,000 psig and 25° C. and a flowrate of 1 mL/min.

The results of CBD-II-57 are shown in Table 12. The biomass batch number and run specifications as well as input and some output information are listed in the first 15 rows of page 1 of Table 2. For example, 20.00 grams of undried and unground biomass was loaded and 16.75 grams were recovered after extraction. The 3.250 grams loss was a combination of cannabinoids and biomass analytes as well as moisture. Based on the measured total biomass extracted of 1.021 g, the moisture removed was 2.229 g or 11.15% (the average moisture content according to RTI data sheet was 8.8+0.03% (n=3); in APH-1403-01, we experimentally measured a value of 7.74%). However, the silica gained 1.43 g which could be either extract, moisture or both.

The first page of Table 12 lists each fraction number in the first column, the time of each fraction in column 2, the CO2 and methanol content in columns 3 and 4 respectively, the color and volume of each fraction in columns 4 and 5 respectively, and the biomass extracted and yield in columns 6 and 7 respectively. The biomass extracted is based on the dry weight analysis of 2 mL of each fraction in triplicate multiplied by the measured volume. The percent yield is the biomass extracted divided by the original biomass weight multiplied by 100. The overall biomass extracted yield was 4.156%. The last row of Table 12, page 1 lists the extract of the spent biomass. During unloading the extractor, three samples of spent biomass were each extracted from the top, middle and bottom of the extractor. Each of these 9 samples was then extracted in hot methanol following SOP No. APH-1403-01. Dry weight analyses were performed in triplicate on each of the 9 extracts and averaged to be 3.07 mg.

TABLE 12
Page 1: CBD-II-57: SFS-CXP of high CBD content Cannabis sativa [unground and unheated]:
Extraction with SFS CO₂ at 3,000 psig and 50° C., Deposition on Silica [heat activated]
followed by Supercitical Fluid Chromatography using SFS CO₂:methanol at 3,000 psig and 25° C.

| Experiment No. | CBD-II-57 | Page 1 | | | | |
|---|---|---|---|---|---|---|
| Date of Experiment: | 15 Jul. 16 | | | | | |
| Designed by: | TC | Performed by: | KOL, WAB | | | |
| Biomass: | Cannabis sativa | Batch No: | RTI-14039-0316-60 | #2 | | |
| | | CB Content of Non-derivatized Extracts: | | D9-THC (%) | 4.00 | CB [symbol] |
| | | CB Content of Derivatized Extracts: | | D9-THC (%) | 0.57 | CB [symbol] |
| | | CB Content -APH-1501-04-48 | | D9-THC (%) | 0.31 | D9 [symbol] |
| Pressure (psig): | 3000 | Preparation: | Biomass was directly packed into extraction column. | | | |
| Temperature (° C.): | 50 | | Not dried or ground. | | | |
| Flowrate (mL/min): | 60 | | | | | |
| BPR Flush (mL/min): | 2 | Procedure: | Aphios SOP No. APH-S0201 | | Analysis: | Aph |
| Original Biomass (g): | 20.00 | | Aphios SOP No. APH-EQ-015 | | | Aph |
| SFS Extracted Biomass (g): | 16.75 | | | | | |

| | | | |
|---|---|---|---|
| Biomass Extracted (mg): | 3250 | Equipment: | SFS-CXP Bench-Top Equipment |
| Original Silica (g): | 23.00 | Silica post-exp (g): | 24.43 |

| | | | | | | Extracted | | |
|---|---|---|---|---|---|---|---|---|
| | Time | CO2 | MeOH | | Vol. | Sample | *Yield | |
| CBD-II-57 | (min) | (% I) | (%) | Color | (mL) | (mg) | (%) | |
| 1 | 20 | 100 | 0 | cloudy/wheat | 73.00 | 7.91 | 0.0395 | Extraction Only |
| 2 | 20 | 100 | 0 | cloudy/white | 65.00 | 6.77 | 0.0339 | Extraction Only |
| 3 | 20 | 100 | 0 | cloudy/white | 67.00 | 5.58 | 0.0279 | Extraction Only |
| 4 | 20 | 100 | 0 | cloudy/white | 66.00 | 7.43 | 0.0371 | Extraction Only |
| 5 | 20 | 100 | 0 | cloudy/whitish | 69.00 | 4.60 | 0.0230 | Extraction Only |
| 6 | 20 | 100 | 0 | cloudy/whitish | 67.00 | 7.82 | 0.0391 | Extraction Only |
| 7 | 20 | 100 | 0 | cloudy/pale yellow | 64.00 | 8.80 | 0.0440 | Extraction Only |
| 8 | 20 | 100 | 0 | cloudy/pale yellow | 71.00 | 12.72 | 0.0636 | Extraction Only |
| 9 | 20 | 100 | 0 | hazy/yellowish | 65.00 | 7.85 | 0.0393 | Extraction Only |
| 10 | 20 | 100 | 0 | hazy/yellowish | 69.00 | 6.04 | 0.0302 | Extraction Only |
| 11 | 20 | 100 | 0 | hazy/yellowish | 69.00 | 5.18 | 0.0259 | Extraction Only |
| 12 | 20 | 100 | 0 | pale yellow | 70.00 | 4.67 | 0.0233 | Extraction Only |
| 13 | 20 | 100 | 0 | pale yellow | 68.00 | 4.53 | 0.0227 | Extraction Only |
| 14 | 20 | 100 | 0 | hazy/pale yellowish | 68.00 | 4.53 | 0.0227 | Extraction Only |
| 15 | 20 | 100 | 0 | clear/yellow | 66.00 | 4.95 | 0.0248 | Extraction Only |
| 16 | 20 | 100 | 0 | clear/yellow | 71.00 | 6.80 | 0.0340 | Extraction Only |
| 17 | 20 | 100 | 0 | clear/yellow | 70.00 | 7.88 | 0.0394 | Extraction Only |
| 18 | 20 | 100 | 0 | clear/yellow | 71.00 | 10.06 | 0.0503 | Extraction Only |
| 19 | 20 | 100 | 0 | clear/yellow | 72.00 | 6.90 | 0.0345 | Extraction Only |
| 20 | 20 | 100 | 0 | clear/yellow | 72.00 | 7.20 | 0.0360 | Chromatography |
| 21 | 20 | 99.5 | 0.5 | clear/yellow | 75.00 | 25.97 | 0.1298 | Chromatography |
| 22 | 20 | 99 | 1 | clear/yellow | 72.00 | 25.50 | 0.1275 | Chromatography |
| 23 | 20 | 98.5 | 1.5 | clear/yellow | 75.00 | 269.08 | 1.3453 | Chromatography |
| 24 | 20 | 98 | 2 | hazy/gold | 75.00 | 371.68 | 1.9578 | Chromatography |
| 25 | 20 | 100 | 0 | hazy/pale yellowish | 75.00 | 68.75 | 0.3438 | Chromatography |
| 26 | 20 | 100 | 0 | yellowish | 69.00 | 9.20 | 0.0460 | Chromatography |
| 27 | 20 | 60 | 40 | greenish gold | 128.00 | 90.67 | 0.4533 | Chromatography |
| 28 | 10 | 100 | 0 | hazy/yellowish | 75.00 | 3.13 | 0.0156 | Chromatography |
| 29 | 10 | 0 | 0 | hazy/pale yellowish | 33.00 | 2.89 | 0.0144 | Decompression |
| 30 | 20 | 0 | 0 | hazy/pale yellowish | 33.00 | 5.23 | 0.0261 | Decompression |
| 31 | 20 | 0 | 100 | cloudy/whitish | 205.00 | 11.10 | 0.0555 | Sep Wash |
| TOTAL | 160 | N/A | N/A | | | 1,021.26 | 5.106 | |
| Spent | 30.10 | 0 | 100 | green/bm green | 25.00 | 3.07 | 0.015 | |
| Biomass | | | | | | | | |

[symbol]

indicates data missing or illegible when filed

The second page of Table 12 lists the HPLC analyses of each fraction following SOP No. APH-EQ-015 in triplicate. The average absorbance of CBDA, CBD, THC, CBN and THCA are listed in columns 2 through 6 for each fraction and spent biomass in the last row. The concentrations of CBDA, CBD, THC, CBN and THCA in mg/mL, computed from standard curves in APH-1501-01 are then listed in columns 7 through 11.

TABLE 12
Page 2: CBD-II-57: SFS-CXP of high CBD content Cannabis sativa [unground
and unheated]: Extraction with SFS CO₂ at 3,000 psig and 50° C.,
Deposition on Silica [heat activated] followed by Supercitical
Fluid Chromatography using SFS CO₂:methanol at
3,000 psig and 25° C.
Experiment No. CBD-II-57 Page: 2

| | CBDA | CBD | THC | CBN | THCA | CBDA | CBD | THC | CBN | THCA | |
|---|---|---|---|---|---|---|---|---|---|---|---|
| CBD-II-57 | (AU) | (AU) | (AU) | (AU) | (AU) | (mg/mL) | (mg/mL) | (mg/mL) | (mg/mL) | (mg/mL) | |
| 1 | 0 | 0 | 0 | 0 | 0 | 0.000 | 0.000 | 0.000 | 0.000 | 0.000 | Extraction |
| | | | | | | | | | | | Only |
| 2 | 0 | 0 | 0 | 0 | 0 | 0.000 | 0.000 | 0.000 | 0.000 | 0.000 | Extraction |
| | | | | | | | | | | | Only |
| 3 | 0 | 0 | 0 | 0 | 0 | 0.000 | 0.000 | 0.000 | 0.000 | 0.000 | Extraction |
| | | | | | | | | | | | Only |
| 4 | 0 | 0 | 0 | 0 | 0 | 0.000 | 0.000 | 0.000 | 0.000 | 0.000 | Extraction |
| | | | | | | | | | | | Only |
| 5 | 0 | 0 | 0 | 0 | 0 | 0.000 | 0.000 | 0.000 | 0.000 | 0.000 | Extraction |
| | | | | | | | | | | | Only |
| 6 | 0 | 0 | 0 | 0 | 0 | 0.000 | 0.000 | 0.000 | 0.000 | 0.000 | Extraction |
| | | | | | | | | | | | Only |
| 7 | 0 | 42,276 | 0 | 0 | 0 | 0.000 | 0.125 | 0.000 | 0.000 | 0.000 | Extraction |
| | | | | | | | | | | | Only |
| 8 | 0 | 62,106 | 0 | 0 | 0 | 0.000 | 0.184 | 0.000 | 0.000 | 0.000 | Extraction |
| | | | | | | | | | | | Only |
| 9 | 0 | 46,993 | 0 | 0 | 0 | 0.000 | 0.139 | 0.000 | 0.000 | 0.000 | Extraction |
| | | | | | | | | | | | Only |
| 10 | 0 | 0 | 0 | 0 | 0 | 0.000 | 0.000 | 0.000 | 0.000 | 0.000 | Extraction |
| | | | | | | | | | | | Only |
| 11 | 0 | 0 | 0 | 0 | 0 | 0.000 | 0.000 | 0.000 | 0.000 | 0.000 | Extraction |
| | | | | | | | | | | | Only |
| 12 | 0 | 0 | 0 | 0 | 0 | 0.000 | 0.000 | 0.000 | 0.000 | 0.000 | Extraction |
| | | | | | | | | | | | Only |
| 13 | 0 | 0 | 0 | 90,611 | 0 | 0.000 | 0.000 | 0.000 | 0.006 | 0.000 | Extraction |
| | | | | | | | | | | | Only |
| 14 | 0 | 0 | 0 | 107,346 | 0 | 0.000 | 0.000 | 0.000 | 0.007 | 0.000 | Extraction |
| | | | | | | | | | | | Only |
| 15 | 0 | 0 | 0 | 131,662 | 0 | 0.000 | 0.000 | 0.000 | 0.008 | 0.000 | Extraction |
| | | | | | | | | | | | Only |
| 16 | 0 | 0 | 0 | 124,055 | 0 | 0.000 | 0.000 | 0.000 | 0.008 | 0.000 | Extraction |
| | | | | | | | | | | | Only |
| 17 | 0 | 0 | 0 | 157,593 | 0 | 0.000 | 0.000 | 0.000 | 0.010 | 0.000 | Extraction |
| | | | | | | | | | | | Only |
| 18 | 0 | 0 | 47,684 | 165,162 | 0 | 0.000 | 0.000 | 0.086 | 0.010 | 0.000 | Extraction |
| | | | | | | | | | | | Only |
| 19 | 0 | 0 | 0 | 141,058 | 0 | 0.000 | 0.000 | 0.000 | 0.009 | 0.000 | Extraction |
| | | | | | | | | | | | Only |
| 20 | 0 | 0 | 0 | 109,260 | 0 | 0.000 | 0.000 | 0.000 | 0.007 | 0.000 | Chromatog- |
| | | | | | | | | | | | raphy |
| 21 | 0 | 32,037 | 194,539 | 352,337 | 0 | 0.000 | 0.095 | 0.270 | 0.022 | 0.000 | Chromatog- |
| | | | | | | | | | | | raphy |
| 22 | 0 | 76,576 | 164,376 | 276,624 | 0 | 0.000 | 0.227 | 0.216 | 0.017 | 0.000 | Chromatog- |
| | | | | | | | | | | | raphy |
| 23 | 17,634,980 | 0 | 469,106 | 377,198 | 14,997 | 5.778 | 0.000 | 0.652 | 0.023 | 0.004 | Chromatog- |
| | | | | | | | | | | | raphy |
| 24 | 14,082,010 | 0 | 0 | 329,014 | 18,832,406 | 4.615 | 0.000 | 0.000 | 0.020 | 4.515 | Chromatog- |
| | | | | | | | | | | | raphy |
| 25 | 855,135 | 0 | 0 | 0 | 2,243,698 | 0.280 | 0.000 | 0.000 | 0.000 | 0.538 | Chromatog- |
| | | | | | | | | | | | raphy |
| 26 | 297,134 | 0 | 0 | 0 | 161,003 | 0.097 | 0.000 | 0.000 | 0.000 | 0.039 | Chromatog- |
| | | | | | | | | | | | raphy |
| 27 | 513,780 | 0 | 0 | 83,589 | 0 | 0.168 | 0.000 | 0.000 | 0.005 | 0.000 | Chromatog- |
| | | | | | | | | | | | raphy |
| 28 | 0 | 0 | 0 | 0 | 0 | 0.000 | 0.000 | 0.000 | 0.000 | 0.000 | Chromatog- |
| | | | | | | | | | | | raphy |
| 29 | 180,538 | 0 | 0 | 0 | 0 | 0.053 | 0.000 | 0.000 | 0.000 | 0.000 | Chromatog- |
| | | | | | | | | | | | raphy |
| 30 | 161,254 | 0 | 0 | 0 | 0 | 0.053 | 0.000 | 0.000 | 0.000 | 0.000 | Chromatog- |
| | | | | | | | | | | | raphy |
| 31 | 0 | 0 | 0 | 0 | 0 | 0.000 | 0.000 | 0.000 | 0.000 | 0.000 | Sep Wash |
| TOTAL | N/A | N/A | N/A | N/A | N/A | 11.05 | 0.77 | 1.20 | 0.15 | 5.10 | |
| Spent | 488,895 | 0 | 0 | 0 | 553,772 | 0.16 | 0.00 | 0.00 | 0.000 | 0.13 | |
| Biomass | | | | | | | | | | | |

The third page of Table 12 lists the computed masses in mg of CBDA, CBD, THC, CBN and THCA in columns 2 through 6 for each fraction and spent biomass in the second to last row. The totals of each column are summed and divided by the biomass load multiplied by 100 to define the yield of each cannabinoid. The cannabinoids recovered are then computed by dividing the yields by the average cannabinoids measured by hot methanol extraction following SOP No. APH-1403-01. The absolute purities are then computed through dividing the mass of each fraction by the biomass extracted multiplied by 100, and listed in columns 7 through 11.

TABLE 12
Page 3: CBD-II-57: SFS-CXP of high CBD content Cannabis sativa [unground
and unheated]: Extraction with SFS CO₂ at 3,000 psig and 50° C.,
Deposition on Silica [heat activated] followed by Supercitical
Fluid Chromatography using SFS CO₂:methanol at 3,000 psig and 25° C.
Experiment No. CBD-II-57 Page 3

| | CBDA | CBD | THC | CBN | THCA | CBDA | CBD | THC | CBN | THCA | |
|---|---|---|---|---|---|---|---|---|---|---|---|
| CBD-II-57 | (mg) | (mg) | (mg) | (mg) | (mg) | (% AP) | (% AP) | (% AP) | (% AP) | (% AP) | |
| 1 | 0.00 | 0.00 | 0.00 | 0.00 | 0.00 | 0.00 | 0.00 | 0.00 | 0.00 | 0.00 | Extraction |
| | | | | | | | | | | | Only |
| 2 | 0.00 | 0.00 | 0.00 | 0.00 | 0.00 | 0.00 | 0.00 | 0.00 | 0.00 | 0.00 | Extraction |
| | | | | | | | | | | | Only |
| 3 | 0.00 | 0.00 | 0.00 | 0.00 | 0.00 | 0.00 | 0.00 | 0.00 | 0.00 | 0.00 | Extraction |
| | | | | | | | | | | | Only |
| 4 | 0.00 | 0.00 | 0.00 | 0.00 | 0.00 | 0.00 | 0.00 | 0.00 | 0.00 | 0.00 | Extraction |
| | | | | | | | | | | | Only |
| 5 | 0.00 | 0.00 | 0.00 | 0.00 | 0.00 | 0.00 | 0.00 | 0.00 | 0.00 | 0.00 | Extraction |
| | | | | | | | | | | | Only |
| 6 | 0.00 | 0.00 | 0.00 | 0.00 | 0.00 | 0.00 | 0.00 | 0.00 | 0.00 | 0.00 | Extraction |
| | | | | | | | | | | | Only |
| 7 | 0.00 | 8.02 | 0.00 | 0.00 | 0.00 | 0.00 | 91.14 | 0.00 | 0.00 | 0.00 | Extraction |
| | | | | | | | | | | | Only |
| 8 | 0.00 | 13.07 | 0.00 | 0.00 | 0.00 | 0.00 | 102.75 | 0.00 | 0.00 | 0.00 | Extraction |
| | | | | | | | | | | | Only |
| 9 | 0.00 | 9.05 | 0.00 | 0.00 | 0.00 | 0.00 | 115.28 | 0.00 | 0.00 | 0.00 | Extraction |
| | | | | | | | | | | | Only |
| 10 | 0.00 | 0.00 | 0.00 | 0.00 | 0.00 | 0.00 | 0.00 | 0.00 | 0.00 | 0.00 | Extraction |
| | | | | | | | | | | | Only |
| 11 | 0.00 | 0.00 | 0.00 | 0.00 | 0.00 | 0.00 | 0.00 | 0.00 | 0.00 | 0.00 | Extraction |
| | | | | | | | | | | | Only |
| 12 | 0.00 | 0.00 | 0.00 | 0.00 | 0.00 | 0.00 | 0.00 | 0.00 | 0.00 | 0.00 | Extraction |
| | | | | | | | | | | | Only |
| 13 | 0.00 | 0.00 | 0.00 | 0.38 | 0.00 | 0.00 | 0.00 | 0.00 | 8.37 | 0.00 | Extraction |
| | | | | | | | | | | | Only |
| 14 | 0.00 | 0.00 | 0.00 | 0.45 | 0.00 | 0.00 | 0.00 | 0.00 | 9.92 | 0.00 | Extraction |
| | | | | | | | | | | | Only |
| 15 | 0.00 | 0.00 | 0.00 | 0.53 | 0.00 | 0.00 | 0.00 | 0.00 | 10.81 | 0.00 | Extraction |
| | | | | | | | | | | | Only |
| 16 | 0.00 | 0.00 | 0.00 | 0.54 | 0.00 | 0.00 | 0.00 | 0.00 | 7.97 | 0.00 | Extraction |
| | | | | | | | | | | | Only |
| 17 | 0.00 | 0.00 | 0.00 | 0.58 | 0.00 | 0.00 | 0.00 | 0.00 | 8.83 | 0.00 | Extraction |
| | | | | | | | | | | | Only |
| 18 | 0.00 | 0.00 | 4.70 | 0.74 | 0.00 | 0.00 | 0.00 | 46.68 | 7.31 | 0.00 | Extraction |
| | | | | | | | | | | | Only |
| 19 | 0.00 | 0.00 | 0.00 | 0.63 | 0.00 | 0.00 | 0.00 | 0.00 | 9.07 | 0.00 | Extraction |
| | | | | | | | | | | | Only |
| 20 | 0.00 | 0.00 | 0.00 | 0.48 | 0.00 | 0.00 | 0.00 | 0.00 | 6.73 | 0.00 | Chromatog- |
| | | | | | | | | | | | raphy |
| 21 | 0.00 | 7.22 | 20.55 | 1.65 | 0.00 | 0.00 | 27.79 | 79.16 | 6.35 | 0.00 | Chromatog- |
| | | | | | | | | | | | raphy |
| 22 | 0.00 | 16.34 | 16.45 | 1.23 | 0.00 | 0.00 | 64.09 | 60.59 | 4.81 | 0.00 | Chromatog- |
| | | | | | | | | | | | raphy |
| 23 | 433.44 | 0.00 | 48.91 | 1.74 | 0.27 | 161.09 | 0.00 | 18.18 | 0.85 | 0.10 | Chromatog- |
| | | | | | | | | | | | raphy |
| 24 | 346.12 | 0.00 | 0.00 | 1.52 | 338.86 | 93.15 | 0.00 | 0.00 | 0.41 | 91.14 | Chromatog- |
| | | | | | | | | | | | raphy |
| 25 | 21.02 | 0.00 | 0.00 | 0.00 | 40.35 | 30.67 | 0.00 | 0.00 | 0.00 | 58.69 | Chromatog- |
| | | | | | | | | | | | raphy |
| 26 | 6.72 | 0.00 | 0.00 | 0.00 | 2.66 | 73.03 | 0.00 | 0.00 | 0.00 | 28.95 | Chromatog- |
| | | | | | | | | | | | raphy |
| 27 | 21.55 | 0.00 | 0.00 | 0.66 | 0.00 | 23.77 | 0.00 | 0.00 | 0.73 | 0.00 | Chromatog- |
| | | | | | | | | | | | raphy |
| 28 | 0.00 | 0.00 | 0.00 | 0.00 | 0.00 | 0.00 | 0.00 | 0.00 | 0.00 | 0.00 | Chromatog- |
| | | | | | | | | | | | raphy |
| 29 | 1.74 | 0.00 | 0.00 | 0.00 | 0.00 | 60.13 | 0.00 | 0.00 | 0.00 | 0.00 | Decom- |
| | | | | | | | | | | | pression |
| 30 | 1.74 | 0.00 | 0.00 | 0.00 | 0.00 | 33.38 | 0.00 | 0.00 | 0.00 | 0.00 | Decom- |
| | | | | | | | | | | | pression |
| 31 | 0.00 | 0.00 | 0.00 | 0.00 | 0.00 | 0.00 | 0.00 | 0.00 | 0.00 | 0.00 | Sep Wash |
| TOTAL | 832.33 | 53.70 | 89.61 | 11.23 | 381.94 | | | | | | |
| YIELD (%) | 4.16 | 0.27 | 0.45 | 0.86 | 1.91 | N/A | N/A | N/A | N/A | N/A | |
| Spent | 0.02 | 0.00 | 0.00 | 0.00 | 0.02 | N/A | N/A | N/A | N/A | N/A | |
| Biom. (%) | | | | | | | | | | | |
| RECOVERY (%) | 88.55 | 127.87 | 144.53 | 140.38 | 74.31 | | | | | | |

*Yield = (Extracted Sample/Original Biomass)*100
** Recovery = (Yield/Aphios Assayed Content)*100

The fourth page of Table 12 is a summary of the prior 3 pages and lists the fraction number, time, CO2 and methanol concentrations as well as the mass and absolute purities of cannabinoids extracted and purified.

TABLE 12
Page 4: CBD-II-57: SFS-CXP of high CBD content Cannabis sativa [unground
and unheated]: Extraction with SFS CO₂ at 3,000 psig and 50° C.,
Deposition on Silica [heat activated] followed by Supercitical
Fluid Chromatography using SFS CO₂:methanol at 3,000 psig and 25° C.
Experiment No. CBD-II-57 Page 4

| | Time | CO2 | MeOH | CBDA | CBD | THC | CBN | THCA | CBDA | CBD | THC | CBN | THCA | |
|---|---|---|---|---|---|---|---|---|---|---|---|---|---|---|
| CBD-II-57 | (min) | (%) | (%) | (mg) | (mg) | (mg) | (mg) | (mg) | (% AP) | (% AP) | (% AP) | (% AP) | (% AP) | |
| 1 | 20 | 100 | 0 | 0.00 | 0.00 | 0.00 | 0.00 | 0.00 | 0.00 | 0.00 | 0.00 | 0.00 | 0.00 | Extraction |
| | | | | | | | | | | | | | | Only |
| 2 | 20 | 100 | 0 | 0.00 | 0.00 | 0.00 | 0.00 | 0.00 | 0.00 | 0.00 | [symbol] | 0.00 | 0.00 | Extraction |
| | | | | | | | | | | | | | | Only |
| 3 | 20 | 100 | 0 | 0.00 | 0.00 | 0.00 | 0.00 | 0.00 | 0.00 | 0.00 | [symbol] | 0.00 | 0.00 | Extraction |
| | | | | | | | | | | | | | | Only |
| 4 | 20 | 100 | 0 | 0.00 | 0.00 | 0.00 | 0.00 | 0.00 | 0.00 | 0.00 | 0.00 | 0.00 | 0.00 | Extraction |
| | | | | | | | | | | | | | | Only |
| 5 | 20 | 100 | 0 | 0.00 | 0.00 | 0.00 | 0.00 | 0.00 | 0.00 | 0.00 | 0.00 | 0.00 | 0.00 | Extraction |
| | | | | | | | | | | | | | | Only |
| 6 | 20 | 100 | 0 | 0.00 | 0.00 | 0.00 | 0.00 | 0.00 | 0.00 | 0.00 | 0.00 | 0.00 | 0.00 | Extraction |
| | | | | | | | | | | | | | | Only |
| 7 | 20 | 100 | 0 | 0.00 | 0.00 | 0.00 | 0.00 | 0.00 | 0.00 | 100.00 | 0.00 | 0.00 | 0.00 | Extraction |
| | | | | | | | | | | | | | | Only |
| 8 | 20 | 100 | 0 | 0.00 | 13. [symbol] | 0.00 | 0.00 | 0.00 | 0.00 | 100.00 | 0.00 | 0.00 | 0.00 | Extraction |
| | | | | | | | | | | | | | | Only |
| 9 | 20 | 100 | 0 | 0.00 | 0.00 | 0.00 | 0.00 | 0.00 | 0.00 | 100.00 | 0.00 | 0.00 | 0.00 | Extraction |
| | | | | | | | | | | | | | | Only |
| 10 | 20 | 100 | 0 | 0.00 | 0.00 | 0.00 | 0.00 | 0.00 | 0.00 | 0.00 | 0.00 | 0.00 | 0.00 | Extraction |
| 11 | 20 | 100 | 0 | 0.00 | 0.00 | 0.00 | 0.00 | 0.00 | 0.00 | 0.00 | 0.00 | 0.00 | 0.00 | Extraction |
| | | | | | | | | | | | | | | Only |
| 12 | 20 | 100 | 0 | 0.00 | 0.00 | 0.00 | 0.00 | 0.00 | 0.00 | 0.00 | 0.00 | 0.00 | 0.00 | Extraction |
| | | | | | | | | | | | | | | Only |
| 13 | 20 | 100 | 0 | 0.00 | 0.00 | 0.00 | [symbol] | 0.00 | 0.00 | 0.00 | 0.00 | 100.00 | 0.00 | Extraction |
| | | | | | | | | | | | | | | Only |
| 14 | 20 | 100 | 0 | 0.00 | 0.00 | 0.00 | [symbol] | 0.00 | 0.00 | 0.00 | 0.00 | 100.00 | 0.00 | Extraction |
| | | | | | | | | | | | | | | Only |
| 15 | 20 | 100 | 0 | 0.00 | 0.00 | 0.00 | [symbol] | 0.00 | 0.00 | 0.00 | 0.00 | 100.00 | 0.00 | Extraction |
| | | | | | | | | | | | | | | Only |
| 16 | 20 | 100 | 0 | 0.00 | 0.00 | 0.00 | 0.00 | 0.00 | 0.00 | 0.00 | 0.00 | 100.00 | 0.00 | Extraction |
| | | | | | | | | | | | | | | Only |
| 17 | 20 | 100 | 0 | 0.00 | 0.00 | 0.00 | 0.00 | 0.00 | 0.00 | 0.00 | 0.00 | 100.00 | 0.00 | Extraction |
| | | | | | | | | | | | | | | Only |
| 18 | 20 | 100 | 0 | 0.00 | 0.00 | [symbol] | [symbol] | 0.00 | 0.00 | 0.00 | [symbol] | [symbol] | 0.00 | Extraction |
| | | | | | | | | | | | | | | Only |
| 19 | 20 | 100 | 0 | 0.00 | 0.00 | 0.00 | 0.00 | 0.00 | 0.00 | 0.00 | 0.00 | 100.00 | 0.00 | Extraction |
| | | | | | | | | | | | | | | Only |
| 20 | 20 | 100 | 0 | 0.00 | 0.00 | 0.00 | 0.00 | 0.00 | 0.00 | 0.00 | 0.00 | 100.00 | 0.00 | Chromatog- |
| | | | | | | | | | | | | | | raphy |
| 21 | 20 | 99.5 | 0.5 | 0.00 | [symbol] | [symbol] | 1. [symbol] | [symbol] | [symbol] | [symbol] | [symbol] | [symbol] | 0.00 | Chromatog- |
| | | | | | | | | | | | | | | raphy |
| 22 | 20 | 99 | 1 | 0.00 | [symbol] | [symbol] | 1. [symbol] | [symbol] | [symbol] | [symbol] | [symbol] | [symbol] | 0.00 | Chromatog- |
| | | | | | | | | | | | | | | raphy |
| 23 | 20 | 98.5 | 1.5 | [symbol] | 0.00 | [symbol] | 1. [symbol] | 0.27 | [symbol] | 0.00 | [symbol] | [symbol] | [symbol] | Chromatog- |
| | | | | | | | | | | | | | | raphy |
| 24 | 20 | 98 | 2 | [symbol] | 0.00 | 0.00 | 1. [symbol] | [symbol] | [symbol] | 0.00 | [symbol] | [symbol] | [symbol] | Chromatog- |
| | | | | | | | | | | | | | | raphy |
| 25 | 20 | 100 | 0 | [symbol] | 0.00 | 0.00 | 0.00 | [symbol] | [symbol] | 0.00 | [symbol] | [symbol] | [symbol] | Chromatog- |
| | | | | | | | | | | | | | | raphy |
| 26 | 20 | 100 | 0 | [symbol] | 0.00 | 0.00 | 0.00 | [symbol] | [symbol] | 0.00 | [symbol] | [symbol] | [symbol] | Chromatog- |
| | | | | | | | | | | | | | | raphy |
| 27 | 20 | 60 | 40 | 21. [symbol] | 0.00 | 0.00 | 0.00 | [symbol] | [symbol] | 0.00 | 0.00 | [symbol] | 0.00 | Chromatog- |
| | | | | | | | | | | | | | | raphy |
| 28 | 10 | 100 | 0 | 0.00 | 0.00 | 0.00 | 0.00 | [symbol] | 0.00 | 0.00 | 0.00 | 0.00 | 0.00 | Chromatog- |
| | | | | | | | | | | | | | | raphy |
| 29 | 10 | 0 | 0 | 1. [symbol] | 0.00 | 0.00 | 0.00 | [symbol] | [symbol] | 0.00 | 0.00 | 0.00 | 0.00 | Decom- |
| | | | | | | | | | | | | | | pression |
| 30 | 20 | 0 | 0 | 1. [symbol] | 0.00 | 0.00 | 0.00 | [symbol] | [symbol] | 0.00 | 0.00 | 0.00 | 0.00 | Decom- |
| | | | | | | | | | | | | | | pression |
| 31 | 20 | 0 | 100 | 0.00 | 0.00 | 0.00 | 0.00 | [symbol] | 0.00 | 0.00 | 0.00 | 0.00 | 0.00 | Sep Wash |
| TOTAL | [symbol] | N/A | N/A | [symbol] | [symbol] | [symbol] | [symbol] | [symbol] | N/A | N/A | N/A | N/A | N/A | |
| YIELD (%) | N/A | N/A | N/A | [symbol] | [symbol] | [symbol] | [symbol] | [symbol] | N/A | N/A | N/A | N/A | N/A | |

[symbol]

indicates data missing or illegible when filed

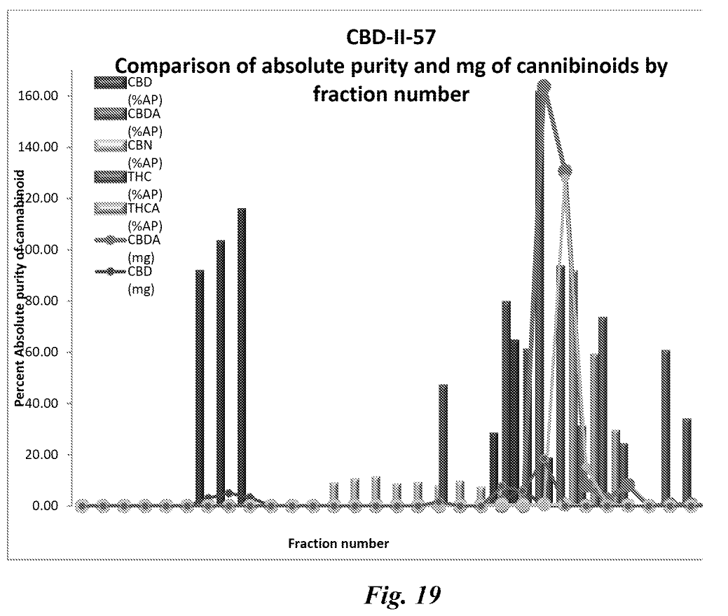

The extraction was very efficient (CBDA recovery of 88.5%, CBD recovery of 127.87%; THC recovery of 144.53%; CBN recovery of 140.38% and THCA recovery of 74.38% based on Aphios analysis of Bag #8 using SOP No. APH-1403-01 in APH-1501-04- 48: 4.7% CBDA, 0.21% CBD, 0.31% THC, 0.04% CBN, and 2.57% THCA). There was poor chromatographic separation between CBDA, THCA and CBN (see fractions 21 to 28 in Table 12 and FIG. 19).

### Example 10: Superfluids™ Extraction and Chromatographic Purifcation of Cannabinoids From Cannabis sativa (CBD-II-59)

Experiment CBD-II-59 was conducted to emulate CBD-II-57 with the exception that the silica was not heated to evaluate if heating had broken down the capacity of the activated silica to retain the carboxylic cannabinoids, CBDA and THCA. The results are summarized in Table 13.

TABLE 13
CBD-II-59: SFS-CXP of high CBD content Cannabis sativa [unground and
unheated]: Extraction with SFS CO₂ at 3,000 psig and 50° C.,
Deposition on Silica [not heat activated] followed by
Supercitical Fluid Chromatography using SFS CO₂:methanol
at 3,000 psig and 25° C.
Experiment No. CBD-II-59 Page 4

| | Time | CO2 | MeOH | CBDA | CBD | THC | CBN | THCA | CBDA | CBD | THC | CBN | THCA | |
|---|---|---|---|---|---|---|---|---|---|---|---|---|---|---|
| CBD-II-59 | (min) | (%) | (%) | (mg) | (mg) | (mg) | (mg) | (mg) | (% AP) | (% AP) | (% AP) | (% AP) | (% AP) | |
| 1 | 20 | 100 | 0 | 0.20 | 0.00 | 0.00 | 0.00 | 0.00 | 100,00 | 0.00 | 0.00 | 0.00 | 0.00 | Extraction |
| | | | | | | | | | | | | | | Only |
| 2 | 20 | 100 | 0 | 0.00 | 43.05 | 0.00 | 0.00 | 0.00 | 0.00 | 100.00 | 0.00 | 0.00 | 0.00 | Extraction |
| | | | | | | | | | | | | | | Only |
| 3 | 20 | 100 | 0 | 0.00 | 19.17 | 9.50 | 1.77 | 0.00 | 0.00 | 62.98 | 31.20 | 5.83 | 0.00 | Extraction |
| | | | | | | | | | | | | | | Only |
| 4 | 20 | 100 | 0 | 0.00 | 3.51 | 38.83 | 5.29 | 0.00 | 0.00 | 7.37 | 81.52 | 11.11 | 0.00 | Extraction |
| | | | | | | | | | | | | | | Only |
| 5 | 20 | 100 | 0 | 0.00 | 0.00 | 1.55 | 0.51 | 0.00 | 0.00 | 0.00 | 75.16 | 24.84 | 0.00 | Extraction |
| | | | | | | | | | | | | | | Only |
| 6 | 20 | 100 | 0 | 0.00 | 0.00 | 0.00 | 0.07 | 0.00 | 0.00 | 0.00 | 0.00 | 100.00 | 0.00 | Extraction |
| | | | | | | | | | | | | | | Only |
| 7 | 20 | 100 | 0 | 0.00 | 0.00 | 0.00 | 0.12 | 0.00 | 0.00 | 0.00 | 0.00 | 100.00 | 0.00 | Extraction |
| | | | | | | | | | | | | | | Only |
| 8 | 20 | 100 | 0 | 0.00 | 0.00 | 0.00 | 0.18 | 0.00 | 0.00 | 0.00 | 0.00 | 100.00 | 0.00 | Extraction |
| | | | | | | | | | | | | | | Only |
| 9 | 20 | 100 | 0 | 1.25 | 0.00 | 0.00 | 0.25 | 0.00 | 83.21 | 0.00 | 0.00 | 16.79 | 0.00 | Extraction |
| | | | | | | | | | | | | | | Only |
| 10 | 20 | 100 | 0 | 4.42 | 0.00 | 0.00 | 0.29 | 0.00 | 93.29 | 0.00 | 0.00 | 6.11 | 0.00 | Extraction |
| 11 | 20 | 100 | 0 | 4.10 | 0.00 | 0.00 | 0.07 | 0.00 | 96.38 | 0.00 | 0.00 | 1.64 | 0.00 | Extraction |
| | | | | | | | | | | | | | | Only |
| 12 | 20 | 100 | 0 | 4.43 | 0.00 | 0.00 | 0.00 | 0.00 | 100.00 | 0.00 | 0.00 | 0.00 | 0.00 | Extraction |
| | | | | | | | | | | | | | | Only |
| 13 | 20 | 100 | 0 | 3.72 | 0.00 | 0.00 | 0.00 | 0.00 | 100.00 | 0.00 | 0.00 | 0.00 | 0.00 | Extraction |
| | | | | | | | | | | | | | | Only |
| 14 | 20 | 100 | 0 | 3.54 | 0.00 | 0.00 | 0.00 | 0.00 | 100.00 | 0.00 | 0.00 | 0.00 | 0.00 | Extraction |
| | | | | | | | | | | | | | | Only |
| 15 | 20 | 100 | 0 | 3.98 | 0.00 | 0.00 | 0.00 | 0.00 | 100.00 | 0.00 | 0.00 | 0.00 | 0.00 | Extraction |
| | | | | | | | | | | | | | | Only |
| 16 | 20 | 100 | 0 | 1.69 | 0.00 | 0.00 | 0.00 | 0.00 | 100.00 | 0.00 | 0.00 | 0.00 | 0.00 | Extraction |
| | | | | | | | | | | | | | | Only |
| 17 | 20 | 100 | 0 | 2.89 | 0.00 | 0.00 | 0.00 | 0.00 | 100.00 | 0.00 | 0.00 | 0.00 | 0.00 | Extraction |
| | | | | | | | | | | | | | | Only |
| 18 | 20 | 100 | 0 | 15.55 | 0.00 | 0.00 | 0.07 | 0.00 | 99.53 | 0.00 | 0.00 | 0.47 | 0.00 | Extraction |
| | | | | | | | | | | | | | | Only |
| 19 | 20 | 100 | 0 | 12.49 | 0.00 | 0.00 | 0.00 | 0.00 | 100.00 | 0.00 | 0.00 | 0.00 | 0.00 | Extraction |
| | | | | | | | | | | | | | | Only |
| 20 | 20 | 100 | 0 | 15.55 | 0.00 | 0.00 | 0.00 | 0.00 | 100.00 | 0.00 | 0.00 | 0.00 | 0.00 | Extraction |
| | | | | | | | | | | | | | | Only |
| 21 | 20 | 99.5 | 0.5 | 40.46 | 0.00 | 0.00 | 0.00 | 0.00 | 100.00 | 0.00 | 0.00 | 0.00 | 0.00 | Chromatog |
| 22 | 20 | 99 | 1 | 51.01 | 0.00 | 0.00 | 0.00 | 0.00 | 100.00 | 0.00 | 0.00 | 0.00 | 0.00 | Chromatog- |
| | | | | | | | | | | | | | | raphy |
| 23 | 20 | 98.5 | 1.5 | 362.18 | 6.20 | 8.64 | 0.28 | 0.00 | 36.00 | 1.64 | 2.29 | 0.07 | 0.00 | Chromatog- |
| | | | | | | | | | | | | | | raphy |
| 24 | 20 | 98 | 2 | 377.38 | 0.00 | 11.52 | 1.62 | 259.85 | 58.02 | 0.00 | 1.79 | 0.25 | 39.95 | Chromatog- |
| | | | | | | | | | | | | | | raphy |
| 25 | 20 | 100 | 0 | 29.92 | 0.00 | 0.00 | 0.64 | 299.01 | 9.08 | 0.00 | 0.00 | 0.19 | 90.73 | Chromatog- |
| | | | | | | | | | | | | | | raphy |
| 26 | 20 | 100 | 0 | 2.72 | 0.00 | 0.00 | 0.00 | 18.87 | 12.82 | 0.00 | 0.00 | 0.00 | 87.38 | Chromatog- |
| | | | | | | | | | | | | | | raphy |
| 27 | 27 | 0 | 0 | 3.56 | 0.00 | 0.00 | 0.00 | 4.11 | 48.37 | 0.00 | 0.00 | 0.00 | 53.63 | Chromatog- |
| | | | | | | | | | | | | | | raphic |
| | | | | | | | | | | | | | | Depressur- |
| | | | | | | | | | | | | | | ization |
| 28 | 9 | 0 | 0 | 0.00 | 0.00 | 0.00 | 0.00 | 0.00 | N/A | N/A | N/A | N/A | N/A | Extractor |
| | | | | | | | | | | | | | | Depressur- |
| | | | | | | | | | | | | | | ization |
| 29 | 29 | 0 | 0 | 0.00 | 0.00 | 0.00 | 0.00 | 0.00 | N/A | N/A | N/A | N/A | N/A | Sep [symbol] |
| TOTAL | 585 | N/A | N/A | 941.05 | 72.73 | 70.14 | 11.17 | 581.84 | 56.12 | 4.34 | N/A | 0.67 | 34.70 | |
| YIELD (%) | N/A | N/A | N/A | 4.71 | 0.36 | 0.35 | 0.06 | 2.91 | N/A | N/A | N/A | N/A | N/A | |

[symbol]

indicates data missing or illegible when filed

The overall recovery efficiencies of CBD-II-59 were excellent: CBDA recovery of 100.11%, CBD recovery of 173.17%; THC recovery of 113.13%; CBN recovery of 139.60% and THCA recovery of 113.20% based on Aphios analysis of Bag #8 using SOP No. APH-1403-01 in APH-1501-04-48: 4.7% CBDA, 0.21% CBD, 0.31% THC, 0.04% CBN, and 2.57% THCA

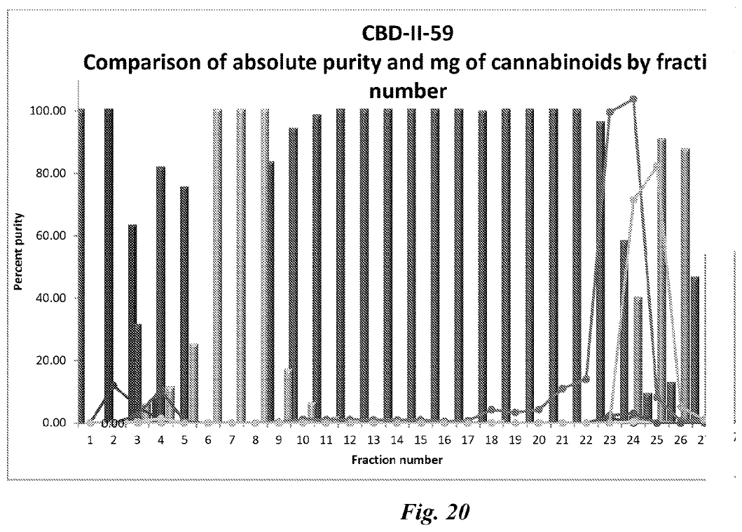

However, there were no improvement in separation efficiencies between THCA, CBDA and CBN as shown in FIG. 20 and very early breakthrough of CBD. The results indicate that separation efficiency was reduced as a result of poor silica performance, possibly caused by not heating the activated silica for 15 hrs at 117° C.

### Example 11: Superfluids™ Extraction and Chromatographic Purifcation of Cannabinoids from Cannabis sativa (CBD-II-61)

Run CBD-II-61 was conducted under similar conditions to CBD-II-59 except the single 40 mL chromatographic column was replaced by two 20 mL chromatographic columns in the bench-top unit. The two chromatographic columns were piped in parallel to increase the deposition surface area in order to maximize cannabinoid retention. In the two-column system, approximately 28 g of unheated, activated silica was packed into the 2 columns about 22% more than the 23 g packed in the single column of CBD-II-59.

Early CBDA breakthrough was observed in Fraction 4 and the experiment was aborted after Fraction 5. Post-experiment flow analysis indicate that the CO2 stream was compromised with ethanol that was left in the line after system reconfiguration, ethanol cleaning and nitrogen blow-out of the lines. The following totals were extracted from the biomass: 357.273 mg CBDA, 416.832 mg CBD, 51.603 mg CBN, 189.378 mg THC and 0.0 mg THCA. Total biomass extracted was 3.56 g or 17.8%.

### Example 12: Superfluids™ Extraction and Chromatographic Purifcation of Cannabinoids from Cannabis sativa (CBD-II-62)

Run CBD-II-62 was conducted as a replacement of the aborted CBD-II-61 and to emulate the successful CBD-II-38 experiment. 14.04 g of unheated silica was loaded in chromatography column A and 13.91 g of unheated silica was loaded in chromatography column B. The two columns were connected in parallel. 20.0 g of unheated, unground biomass was packed into the extractor.

There was significant early breakthrough of CBDA, CBD and CBN in early fractions, and later concomitant breakthrough of CBDA and THCA. These breakthroughs indicate that there was no retention of either the charged or non-charged cannabinoids on the unheated silica. The following totals were extracted from the biomass: 1,156.80 mg CBDA, 65.84 mg CBD, 12.65 mg CBN, 89.92 mg THC and 608.45 mg THCA. The overall recovery efficiencies were: CBDA recovery of 123.04%, CBD recovery of 156.76%; THC recovery of 145.03%; CBN recovery of 158.08% and THCA recovery of 117.99% based on Aphios analysis of Bag #8 using SOP No. APH-1403-01 in APH-1501-04-48: 4.7% CBDA, 0.21% CBD, 0.31% THC, 0.04% CBN, and 2.57% THCA. Total biomass extracted was 4.53 g or 22.7%.

### Example 13: Superfluids™ Extraction and Chromatographic Purifcation of Cannabinoids from Untreated Cannabis sativa (CBD-II-62)

Run CBD-II-63 was conducted as a back-to-back experiment to CBD-II-62 with similar conditions emulating CBD-II-59 and CBD-II-38. The results are remarkably similar to CBD-II-62. The following totals were extracted from the biomass: 933.85 mg CBDA, 104.53 mg CBD, 11.04 mg CBN, 204.01 mg THC and 519.65 mg THCA. The overall recovery efficiencies were: CBDA recovery of 99.35%, CBD recovery of 248.87%; THC recovery of 329.05%; CBN recovery of 137.99% and THCA recovery of 101.10% based on Aphios analysis of Bag #8 using SOP No. APH-1403-01 in APH-1501-04-48: 4.7% CBDA, 0.21% CBD, 0.31% THC, 0.04% CBN, and 2.57% THCA. Total biomass extracted was 3.47 g or 17.4%.

### Example 14: Superfluids™ Extraction and Chromatographic Purifcation of Cannabinoids from Cannabis sativa (CBD-II-67)

Based on the chromatographic separation failures of experiments CBD-II-57, CBD-II-59, CBD-II-61, CBD-II-62 and CBD-II-63, run CBD-II-67 was conducted with dried cannabis biomass and heated silica to remove the presence of water. The two chromatographic columns were also connected in series to increase the length of the chromatographic column. Interestingly, these conditions emulated both of the successful experiments CBD-II-38 and CBD-1-24.

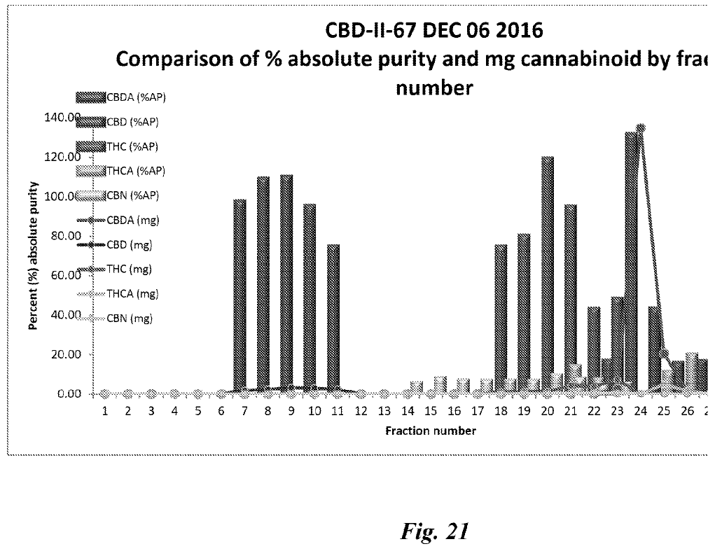

For CBD-II-67, 40 g of dried cannabis biomass (oven-dried at 40° C. for 18 hours), ground into a powder and packed into the 127 mL SS extraction column. The silica was dried at 130° C. for 18 hours in a convective oven and packed into the chromatography columns connected in series. The run conditions are listed in Table 14 and the results are shown in Table 15 and FIG. 21.

CBD-II-67 resulted in excellent early separation of 100% CBD during the extraction step, and later separation of 100% CBDA in the chromatographic step. High purity THC was also eluted prior to CBDA elution; THCA co-eluted later with CBDA. The separation between THCA and CBDA can be improved by smaller step increases of co-solvent concentration.

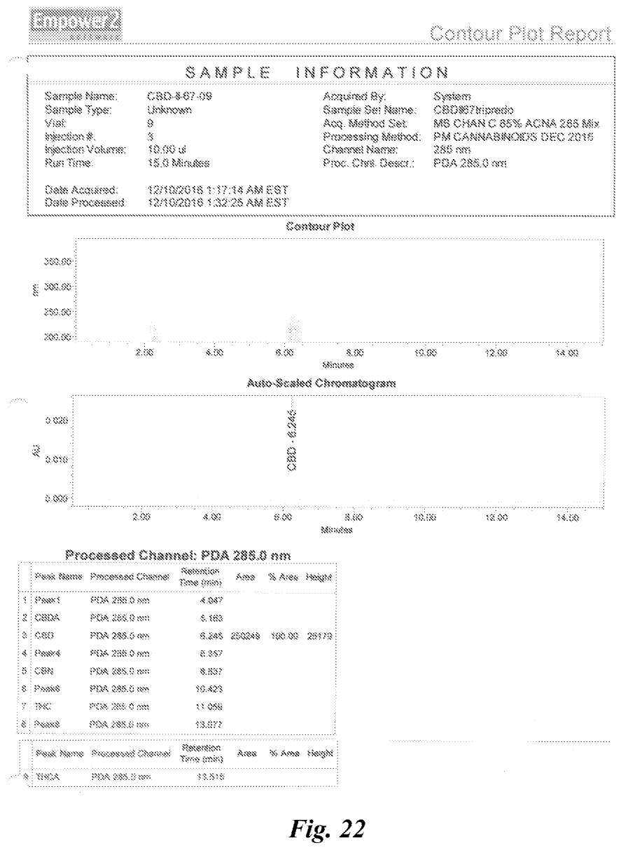

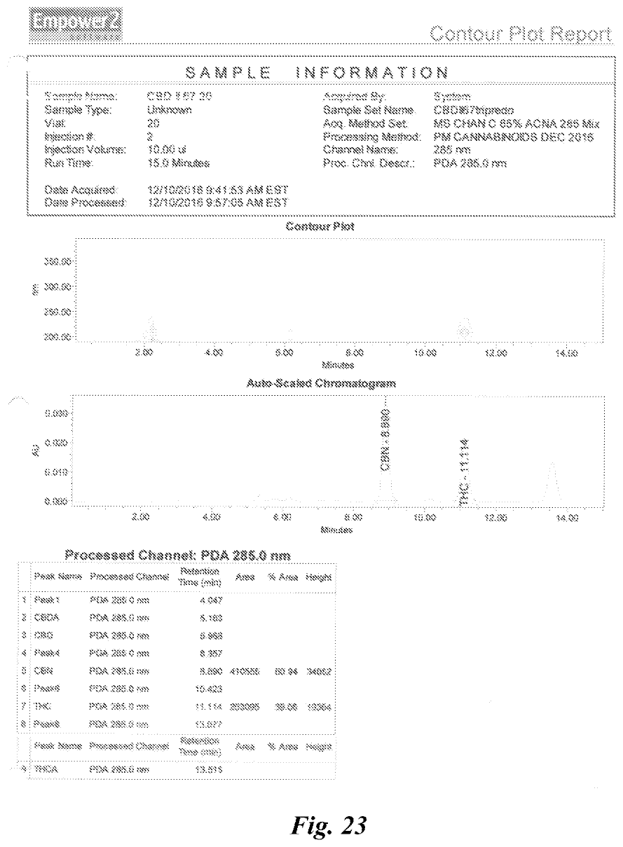

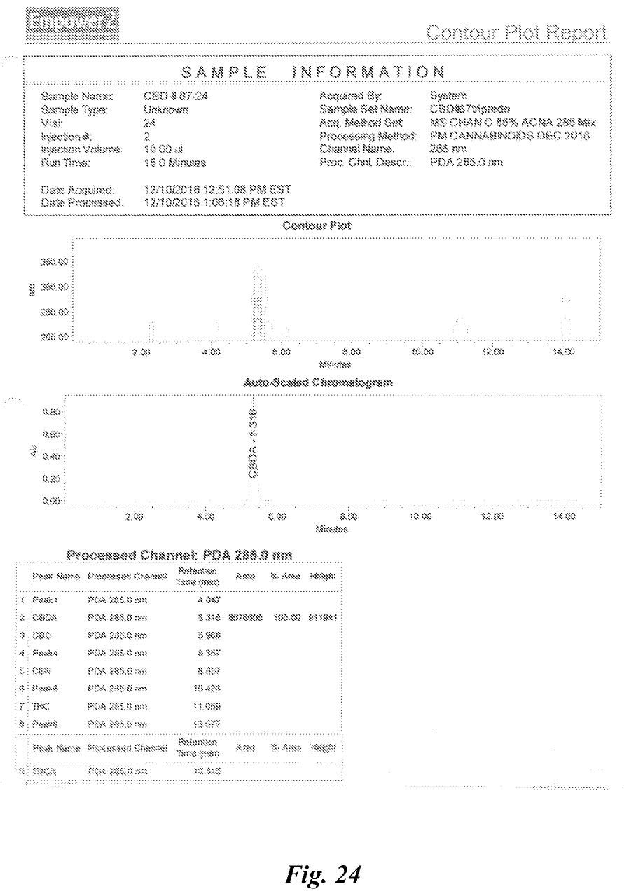

CBD-II-67 produced 206.06 mg of CBD with ˜100% absolute purity (chromatographic scan shown as FIG. 22), 20.26 mg of THC with ˜93.41% absolute purity (chromatographic scan shown as FIG. 23), and ˜2,166.26 mg of CBDA with 100% absolute purity (chromatographic scan shown as FIG. 24).

TABLE 15
CBD-II-67: SFS-CXP of high CBD content Cannabis sativa [ground and
oven-dried at 40° C.]: Extraction with SFS CO₂ at 3,000 psig
and 50° C., Deposition on two Silica columns in series [heat
treated at 130° C.] followed by Supercitical Fluid
Chromatography using SFS CO₂:methanol at 3,000 psig and
25° C. [Like CBD-II-38 and CBD-I-24]
Experiment No. CBD-I-67 Page 4

| | Time | CO2 | MeOH | CBDA | CBD | THC | CBN | THCA | CBDA | CBD | THC | CBN | THCA | |
|---|---|---|---|---|---|---|---|---|---|---|---|---|---|---|
| CBD-I-67 | (min) | (%) | (%) | (mg) | (mg) | (mg) | (mg) | (mg) | (% AP) | (% AP) | (% AP) | (% AP) | (% AP) | |
| 1 | 20 | 100 | 0 | 0.00 | 0.00 | 0.00 | 0.00 | 0.00 | N/A | N/A | N/A | N/A | N/A | Extraction |
| | | | | | | | | | | | | | | Only |
| 2 | 20 | 100 | 0 | 0.00 | 0.00 | 0.00 | 0.00 | 0.00 | N/A | N/A | N/A | N/A | N/A | Extraction |
| | | | | | | | | | | | | | | Only |
| 3 | 20 | 100 | 0 | 0.00 | 0.00 | 0.00 | 0.00 | 0.00 | N/A | N/A | N/A | N/A | N/A | Extraction |
| | | | | | | | | | | | | | | Only |
| 4 | 20 | 100 | 0 | 0.00 | 0.00 | 0.00 | 0.00 | 0.00 | N/A | N/A | N/A | N/A | N/A | Extraction |
| | | | | | | | | | | | | | | Only |
| 5 | 20 | 100 | 0 | 0.00 | 0.00 | 0.00 | 0.00 | 0.00 | N/A | N/A | N/A | N/A | N/A | Extraction |
| | | | | | | | | | | | | | | Only |
| 6 | 20 | 100 | 0 | 0.00 | 0.00 | 0.00 | 0.00 | 0.00 | N/A | N/A | N/A | N/A | N/A | Extraction |
| | | | | | | | | | | | | | | Only |
| 7 | 20 | 100 | 0 | 0.00 | 29.15 | 0.00 | 0.00 | 0.00 | 0.00 | 100.00 | 0.00 | 0.00 | 0.00 | Extraction |
| | | | | | | | | | | | | | | Only |
| 8 | 20 | 100 | 0 | 0.00 | 38.42 | 0.00 | 0.00 | 0.00 | 0.00 | 100.00 | 0.00 | 0.00 | 0.00 | Extraction |
| | | | | | | | | | | | | | | Only |
| 9 | 20 | 100 | 0 | 0.00 | 52.95 | 0.00 | 0.00 | 0.00 | 0.00 | 100.00 | 0.00 | 0.00 | 0.00 | Extraction |
| | | | | | | | | | | | | | | Only |
| 10 | 20 | 100 | 0 | 0.00 | 46.70 | 0.00 | 0.00 | 0.00 | 0.00 | 100.00 | 0.00 | 0.00 | 0.00 | Extraction |
| 11 | 20 | 100 | 0 | 0.00 | 38.82 | 0.00 | 0.00 | 0.00 | 0.00 | 100.00 | 0.00 | 0.00 | 0.00 | Extraction |
| | | | | | | | | | | | | | | Only |
| 12 | 20 | 100 | 0 | 0.00 | 0.00 | 0.00 | 0.00 | 0.00 | N/A | N/A | N/A | N/A | N/A | Extraction |
| | | | | | | | | | | | | | | Only |
| 13 | 20 | 100 | 0 | 0.00 | 0.00 | 0.00 | 0.00 | 0.00 | N/A | N/A | N/A | N/A | N/A | Extraction |
| | | | | | | | | | | | | | | Only |
| 14 | 20 | 100 | 0 | 0.00 | 0.00 | 0.00 | 1.36 | 0.00 | 0.00 | 0.00 | 0.00 | 100.00 | 0.00 | Extraction |
| | | | | | | | | | | | | | | Only |
| 15 | 20 | 100 | 0 | 0.00 | 0.00 | 0.00 | 1.62 | 0.00 | 0.00 | 0.00 | 0.00 | 100.00 | 0.00 | Extraction |
| | | | | | | | | | | | | | | Only |
| 16 | 20 | 100 | 0 | 0.00 | 0.00 | 0.00 | 1.42 | 0.00 | 0.00 | 0.00 | 0.00 | 100.00 | 0.00 | Extraction |
| | | | | | | | | | | | | | | Only |
| 17 | 20 | 100 | 0 | 0.00 | 0.00 | 0.00 | 1.52 | 0.00 | 0.00 | 0.00 | 0.00 | 100.00 | 0.00 | Extraction |
| | | | | | | | | | | | | | | Only |
| 18 | 20 | 100 | 0 | 0.00 | 0.00 | 19.20 | 1.48 | 0.00 | 0.00 | 0.00 | 92.84 | 7.16 | 0.00 | Extraction |
| | | | | | | | | | | | | | | Only |
| 19 | 20 | 100 | 0 | 0.00 | 0.00 | 23.10 | 1.63 | 0.00 | 0.00 | 0.00 | 93.42 | 6.58 | 0.00 | Extraction |
| | | | | | | | | | | | | | | Only |
| 20 | 20 | 100 | 0 | 0.00 | 0.00 | 20.26 | 1.43 | 0.00 | 0.00 | 0.00 | 93.41 | 8.59 | 0.00 | Extraction |
| | | | | | | | | | | | | | | Only |
| 21 | 20 | 100 | 0 | 0.00 | 0.00 | 68.50 | 4.82 | 9.55 | 0.00 | 0.00 | 82.56 | 5.04 | 11.50 | Chromatog |
| 22 | 20 | 99.5 | 0.5 | 0.00 | 0.00 | 60.49 | 5.82 | 9.80 | 0.00 | 0.00 | 79.69 | 7.40 | 12.92 | Chromatog- |
| | | | | | | | | | | | | | | raphy |
| 23 | 20 | 99 | 1 | 39.14 | 0.00 | 116.42 | 10.46 | 7. [symbol] | 22.51 | 0.00 | 66.94 | 6.01 | 4.53 | Chromatog- |
| | | | | | | | | | | | | | | raphy |
| 24 | 20 | 98.5 | 1.5 | 2166.26 | 0.00 | 0.00 | 0.00 | 0.00 | 100.00 | 0.00 | 0.00 | 0.00 | 0.00 | Chromatog- |
| | | | | | | | | | | | | | | raphy |
| 25 | 20 | 98 | 2 | 329.18 | 0.00 | 0.00 | 0.00 | 79.39 | 80.57 | 0.00 | 0.00 | 0.00 | 19.43 | Chromatog- |
| | | | | | | | | | | | | | | raphy |
| 26 | 20 | 100 | 0 | 19.87 | 0.00 | 0.00 | 0.00 | 24.98 | 44.31 | 0.00 | 0.00 | 0.00 | 55.68 | Chromatog- |
| | | | | | | | | | | | | | | raphy |
| 27 | 20 | 100 | 0 | 4.03 | 0.00 | 0.00 | 0.00 | 0.00 | 100.00 | 0.00 | 0.00 | 0.00 | 0.00 | Chromato- |
| | | | | | | | | | | | | | | graphic |
| | | | | | | | | | | | | | | Depressur- |
| | | | | | | | | | | | | | | ization |
| 28 | 20 | 100 | 0 | 7.76 | 0.00 | 0.00 | 0.00 | 0.00 | 100.00 | 0.00 | 0.00 | 0.00 | 0.00 | Extractor |
| | | | | | | | | | | | | | | Depressur- |
| | | | | | | | | | | | | | | ization |
| 29 | 20 | 100 | 0 | 0.00 | 0.00 | 0.00 | 0.00 | 0.00 | N/A | N/A | N/A | N/A | N/A | Sep [symbol] |
| TOTAL | 580 | N/A | N/A | 2566.24 | 206.06 | 307.96 | 31.46 | 131.60 | 79.12 | 6.35 | 9.50 | 0.97 | 4.06 | |
| YIELD (%) | N/A | N/A | N/A | 6.39 | 0.51 | 0.77 | 0.08 | 0.33 | N/A | N/A | N/A | N/A | N/A | |

[symbol]

indicates data missing or illegible when filed

### Example 15: Superfluids™ Extraction and Chromatographic Purifcation of Cannabinoids from Cannabis sativa (CBD-II-78)

To evaluate the significance of heat-drying the silica (which could be challenging at the large-scale), experiment CBD-II-78 was conducted as a replication of CBD-II-67 with heated and dried biomass but not dried silica. The biomass was partly dried in an oven at 40° C.±5° C. for ˜18 h (resulting in 5.74% loss) and then after being packed in the extractor using warm N2 at 60° C. for 4 h and a flowrate of 268 mL/min at 100 psig (resulting in an additional 3.3% water loss). Additionally, ethanol was utilized as the chromatographic elution cosolvent with CO2 instead of methanol.

CBD-II-78 produced 620 mg of CBD with absolute purities between 25% and 78%; 670 mg of Δ9-THC with absolute purities between 16% and 61%; and 1,110 mg of CBDA with absolute purities between 21% and 36% from 40 g of ground, dried cannabis biomass. The data indicated there were significant reductions in absolute purities and separation efficiencies between CBD-II-67 in which the silica was heat-treated to remove moisture and CBD-II-78 in which it was not heat-treated. However, some of this difference could have also be the result of using ethanol rather than methanol as a cosolvent.

### Example 16: In Situ Drying of Cannabis sativa Biomass (CBD-II-80)

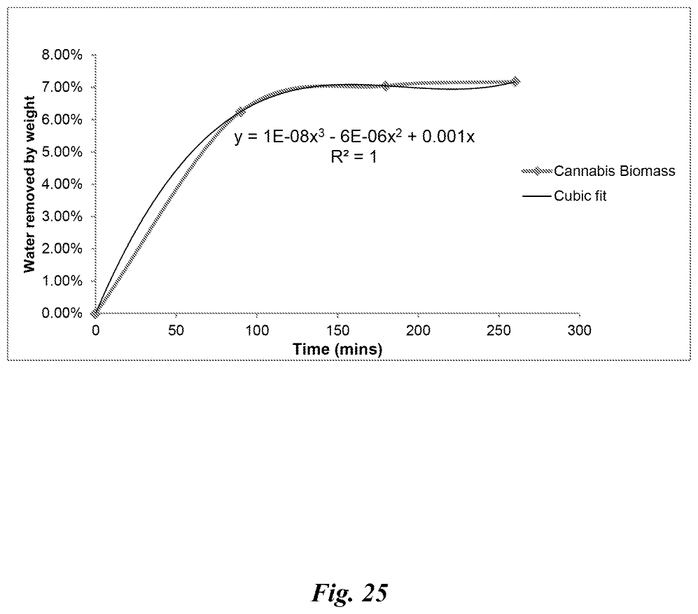

To determine conditions for in-situ drying of ground marijuana biomass after being packed in an extractor, CBD-II-80 was conducted to dry Cannabis biomass in place in the SFS-CXP benchtop extractor using dry N₂ at 60° C. The ground biomass was placed in a stocking and periodically removed and weighed. The weight remained constant after 250 minutes of heating with 10.5 liters per minute of N₂ gas at 60° C. and 100 psig. This resulted in a moisture loss of 7.18%. The drying curve in FIG. 25 was fitted to a polynomial equation y=1E-08x³-6E 06x²+0.001x with an R²=1.

### Example 17: Superfluids™ Extraction and Chromatographic Purifcation of Cannabinoids from CANNABIS SATIVA (CBD-II-81)

CBD-II-81 was conducted on high-CBD content Cannabis biomass, ground and dried in-situ like CBD-II-80 in order to evaluate post-drying extraction efficiency of the biomass and the ability of silica to retain CBDA and THCA after drying of Cannabis biomass with N2 at 60° C. in the extractor. This experiment was conducted to simulate what can be done in the semi-works without prior drying of silica. This experiment was also conducted to evaluate the post- chromatographic elution with methanol rather than ethanol cosolvent as was done in CBD-II-78.

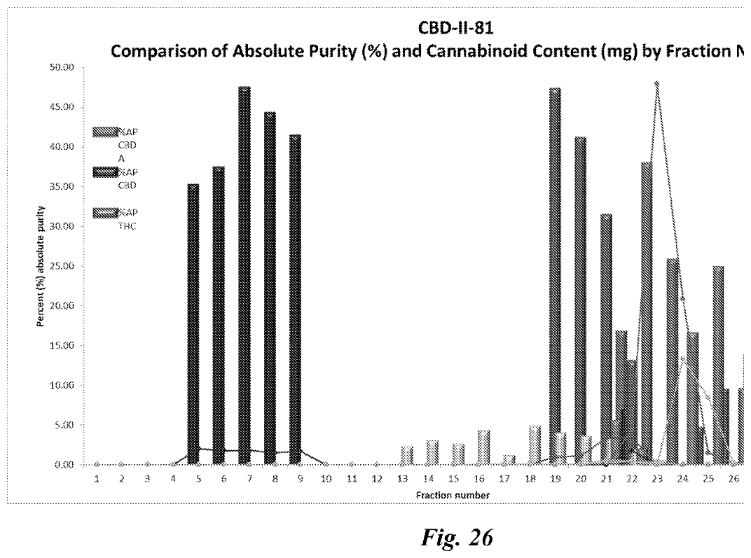

As in the latter experiment, CBD-II-81 was performed with SFS CO₂ at 3,000 psig and 50° C. with deposition on two silica columns in series and chromatographic elution with CO₂ and cosolvent at 3,000 psig and 25° C. The separation profiles in FIG. 26 for CBD-II-81 are remarkably like the separation profiles for CBD-II-67 in FIG. 21. However, CBD-II-81 only produced 160 mg of CBD with absolute purities between 35% and 47%; 134 mg of Δ9-THC with absolute purities between 13% and 47%; and 1,031 mg of CBDA with absolute purities between 26% and 38% from 36.5 g of ground, dried Cannabis biomass.

This data suggests that residual water in the biomass (typical moisture content was measured to be 10.2% but only 7.18% was removed by in-situ drying, leaving a residual of 3% in the biomass) significantly impacts cannabinoid extraction efficiencies.

### Example 18: Superfluids™ Extraction and Chromatographic Purifcation of Cannabinoids from Cannabis sativa (CBD-II-92)

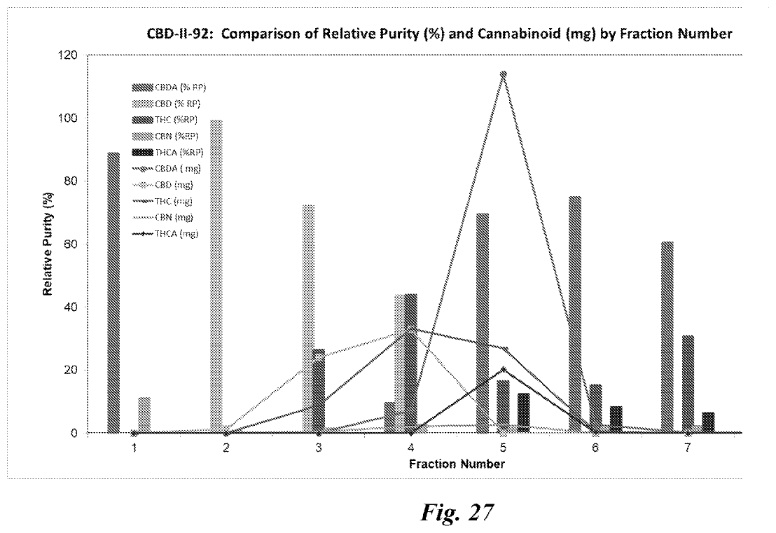

In CBD-II-92, SFS-CXP semi-works (shown in FIG. 4) extraction and chromatographic purification of cannabinoids from high CBD content Cannabis sativa, which was first ground and dried in place with warm air at 40° C., was conducted using SFS CO₂ at 3,000 psig and 50° C. with methanol as a backpressure regulator (BPR) flush solvent in extraction step and co-solvent of CO2 in chromatographic step with activated silica at 3,000 psig and 25° C. [This is a scale-up based on experiments CBD-I-24, CBD-II-38, CBD-II-67 and CBD-II-78, and a re-run of CBD-II-87, CBD-II-88, CBD-II-90, CBD-91]. The results of the scale-up run CBD-II-92 are shown in FIG. 27.

In CBD-II-92, all of the extracted CBD (291.5 g) was produced during the extraction step as anticipated since the neutral CBD will pass through the silica chromatography column. The first CBD fraction (6.5 g) [Fraction No. 2] had a relative purity of 99.15% and an absolute purity of 19.5% and did not contain any 49-THC. Fraction No. 3, the second CBD fraction, contained 120.2 g CBD with a relative purity of 72.2% and an absolute purity of 51.5% and co-eluted with 44.0 g of 49-THC with a relative purity of 26.4% and an absolute purity of 18.8%. Fraction No. 4, the third and largest CBD fraction, contained 164.7 g CBD and co-eluted with an equal quantity of 49-THC and 10.3 g CBN.

The elution of CBD and Δ9-THC was surprising and unexpected since the loaded biomass only contained CBDA (5.088%), Δ9-THCA (2.498%) and CBN (0.083%). The conversions of CBDA to CBD and Δ9-THCA to Δ9-THC were probably caused by the drying of the ground Cannabis sativa biomass with warm compressed air at 40° C., with the conversions being driven by oxidation potential. These conversions can be prevented by using warm compressed nitrogen at 40° C. or even 60° C. for in-situ drying of the ground Cannabis sativa biomass.

CBD-II-92 produced 622.4g of CBDA with relative purities between 10% and 100%, and absolute purities between 2% and 54%. The highest yielding fraction after the start of the chromatographic step was Fraction No. 5 which contained 569.7 g CBDA with a relative purity of 69.6% and an absolute purity of 54%.

Unexpectedly, a small quantity CBDA (0.3 g) with a relative purity of 88% and an absolute purity of 2% was eluted in Fraction No. 1. This was probably caused by the SFS channeling through the silica before it was fully pressurized to operating pressure.

Small quantities of CBDA, Δ9-THC and A9-THCA with trace quantities of CBN were produced in the last 3 chromatographic steps, Fractions Nos. 6, 7 and 8. Thus, while most of the CBDA was eluted in a single fraction, the SFS chromatography can be improved. We anticipate that improvements can be made by better drying of the biomass and silica to remove OH-from binding sites on the silica, and better control of the flowrate and composition of the SFS mobile phase.

The overall yield of CBDA and CBD was 2.55% giving a recovery efficiency of 50.1%, and the overall yield of Δ9-THCA and Δ9-THC was 1.27% giving a recovery efficiency of 50.1%. This data suggests the extraction time should be doubled from 338 minutes to 676 minutes at the operating conditions of pressure, temperature and flowrate utilized in CBD-II-92.

While this invention has been particularly shown and described with references to specific embodiments, figures, detailed description and several examples, it will be understood by those skilled in the art that various changes in form and details may be made therein without departing from the spirit and scope of the invention as defined by the following claims.

What is claimed is:

1. A method of making and separating cannabinoids wherein a mixture of cannabinoids is extracted from Cannabis sativa biomass utilizing first SuperFluids™, wherein the cannabinoids extracted by the first SuperFluids are deposited onto the head of a chromatographic column containing a solid phase and wherein the unabsorbed cannabinoids that flow through the column are collected in separate fractions, and wherein in a second step the extraction column is bypassed, wherein the composition of the SuperFluids™ is changed to form a second SuperFluids™ and wherein the second SuperFluids™ is used to elute and separate the adsorbed cannabinoids from the chromatographic column, wherein the eluted cannabinoids are collected in separate fractions.

2. The method of claim 1 wherein the first SuperFluids™ is carbon dioxide that is held at a temperature in the range of 20-60° C. and at a pressure in the range of 1,000 to 5,000 psig.

3. The method of claim 2 wherein the first SuperFluids™ is carbon dioxide that is held at a temperature of 50° C. and at a pressure of 3,000 psig.

4. The method of claim 1 wherein the second SuperFluids™M is carbon dioxide with a polar modifier that is held at a temperature in the range of 20-60° C. and at a pressure in the range of 1,000 to 5,000 psig.

5. The method of claim 4 wherein the second SuperFluids™ is carbon dioxide with a polar modifier that is held at a temperature of 25° C. and at a pressure of 3,000 psig.

6. The method of claim 4 wherein the preferred modifier is an alcohol such as methanol or ethanol.

7. The method of claim 4 wherein the preferred modifier is methanol.

8. The method of claim 4 wherein the modifier has a molar concentration in the range of 0.1% to 5%.

9. The method of claim 8 wherein the modifier has a molar concentration in the range of 0.1% to 0.5%.

10. The method of claim 4 wherein the SuperFluids™ is a gradient of 99.8% CO₂:0.2% modifier to 98% CO₂:2% modifier.

11. The method of claim 4 wherein the SuperFluids™ is an isocratic mixture of 98.5% CO2 and 1.5% modifier.

12. The method of claim 1 wherein the Cannabis sativa, hemp and Marijuana biomass is ground into a fine powder of 100 to 500 mesh to increase surface are to volume area and increase extraction efficiency.

13. The method of claim 1 wherein the Cannabis sativa, hemp and Marijuana biomass is dried at a low temperature of 25° C. to 60° C. to preserve the integrity of the cannabinoids and to remove the mass transfer resistance of water.

14. The method of claim 13 wherein the Cannabis sativa, hemp and Marijuana biomass is dried with N₂ or air at a low temperature of 25° C. to 60° C.

15. The method of claim 14 wherein the Cannabis sativa, hemp and Marijuana biomass is dried with N₂ at a temperature of 60° C.

16. The method of claim 1 wherein the solid phase in the chromatographic column has a particle size of 10 to 100 microns and consists of C18, C10, C8 or silica.

17. The method of claim 16 wherein the solid phase is silica.

18. The method of claim 16 wherein the solid phase is dehydrated of water and activated for the adsorption of analytes.

19. A method of selecting preferred conditions for extracting cannabinoids from Cannabis sativa and Marijuana using SuperFluids™ by matching the thermodynamic solubility parameters of the SuperFluids™ with thermodynamic solubility parameters of the cannabinoids.

20. An apparatus for making and separating cannabinoids this system consisting of a high pressure syringe or piston pump for neat critical fluid (e.g. CO₂), high pressure or syringe pump for modifier (e.g. ethanol), an extractor for holding the biomass, a chromatographic column for holding the chromatographic separation media, a back-pressure for holding the pressure, a high pressure or syringe flush pump for modifier (e.g. ethanol) to flush and keep clear the back-pressure regulator, and a collection vial 15.

21. (canceled)

22. (canceled)

23. (canceled)

24. (canceled

25. (canceled) rated for an operating pressure of 3,000 psig and temperature of 100° C., wherein the unit is piped to allow several flow paths including: (1) extraction only with near-critical, critical or supercritical fluid with or without a co-solvent: (2) extraction with chromatography through one or both chromatographic columns; (3) chromatography only through one or both chromatographic columns.

26. (canceled)
